(19) United States
(12) Patent Application Publication
(10) Pub. No.: US 2008/0009477 A1
(43) Pub. Date: Jan. 10, 2008

(54) Arylalkyl Ureas As Cb1 Antagonists

(75) Inventors: Alan J. Hutchison, Madison, CT (US); Jun Yuan, Guilford, CT (US)

Correspondence Address: NEUROGEN CORPORATION, 35 NORTHEAST INDUSTRIAL ROAD, BRANFORD, CT, 06405, US

(73) Assignee: NEUROGEN CORPORATION, Branford, CT (US)

(21) Appl. No.: 11/718,421

(22) PCT Filed: Nov. 1, 2005
(86) PCT No.: PCT/US05/39474

§ 371 Date: May 2, 2007

Related U.S. Application Data
(60) Provisional application No. 60/625,234, filed on Nov. 4, 2004.

(51) Int. Cl. A61K 31/55; A61K 31/17; A61K 31/337; A61K 31/352; A61K 31/40; A61P 1/00; A61P 11/06; A61P 25/00; G01N 33/52; A61P 3/04; A61P 19/00; A61P 1/16; A61K 31/551; A61K 31/4164; A61K 31/435; A61K 31/439

(52) U.S. Cl. 514/215; 436/108; 514/221; 514/282; 514/330; 514/397; 514/426; 514/449; 514/456; 514/596; 514/598

(57) ABSTRACT

CB1 antagonists are provided. Such compounds may be used to modulate CB1 activity in vivo or in vitro, and are particularly useful in the treatment of conditions responsive to CB1 receptor modulation in humans, domesticated companion animals and livestock animals, including appetite disorders, obesity and addictive disorders. Pharmaceutical compositions and methods for using them to treat such disorders are provided, as are methods for using ligands for receptor localization studies and various in vitro assays.

### SUMMARY OF THE INVENTION

The present invention provides arylalkyl ureas as CB1 antagonists. Such compounds generally satisfy Formula I: 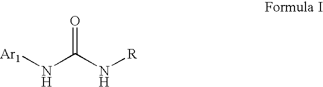
or are a pharmaceutically acceptable salt of such a compound. Within Formula I:
R is:
   (a) (5- or 6-membered heteroaryl)C₁-C₆alkyl or phenylC₁-C₆alkyl, each of which is substituted with from 0 to 4 substituents that are independently chosen from Rₓ; or
   (b) (C₃-C₈cycloalkyl)C₀-C₆alkyl or (3- to 8-membered heterocycloalkyl)C₀-C₆alkyl, each of which is
      (i) optionally fused to a 5- to 7-membered carbocyclic or heterocyclic ring, and
      (ii) optionally substituted, preferably with from 0 to 4 substituents that are independently chosen from Rₓ, or are independently chosen from R_{y};
Ar₁ is phenyl, naphthyl or a 5- to 10-membered heteroaryl, each of which is optionally substituted, and each of which is preferably substituted with from 0 to 4 substituents that are independently chosen from R_{y};
Each Rₓ is independently:
   (a) hydroxy, halogen, amino, cyano, nitro, aminocarbonyl, aminosulfonyl or —COOH;
   (b) a group of the formula L-M₁-Q₁, wherein:
      L is absent or C₀-C₄alkylene;
      M₁ is absent, 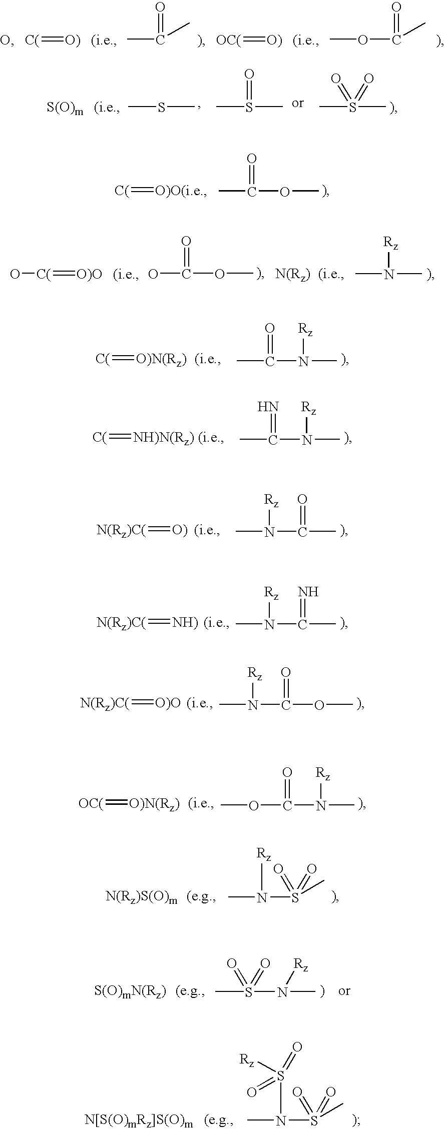
      wherein m is independently selected at each occurrence from 0, 1 and 2; and R_{z} is independently selected at each occurrence from hydrogen, C₁-C₈alkyl and groups that are taken together with Q₁ to form an optionally substituted 4- to 7-membered heterocycle; preferably M₁ is absent, O, OC(═O), O—C(═O)O, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; and
      Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-membered heterocycle, each of which is substituted with from 0 to 3 substituents independently chosen from hydroxy, halogen, amino, cyano, oxo, C₁-C₄alkyl, C₁-C₄alkoxy and C₁-C₄haloalkyl;
      such that, in certain embodiments, Rₓ is preferably not unsubstituted benzyl; or
   (c) taken together with an Rₓ located on an adjacent ring carbon atom to form a fused 5- to 7-membered carbocycle or heterocycle that is optionally substituted, and is preferably substituted with from 0 to 3 substituents independently chosen from hydroxy, halogen, amino, cyano, oxo, C₁-C₄alkyl, C₁-C₄alkoxy, C₁-C₄haloalkyl and C₁-C₄alkylsulfonyl; and
Each R_{Y} is independently:
   (a) hydroxy, halogen, amino, cyano, nitro, aminocarbonyl, aminosulfonyl or —COOH;
   (b) a group of the formula L-M₂-Q₂, wherein:
      L is absent or C₀-C₄alkylene;
      M₂ is absent, O, C(═O), OC(═O), C(═O)O, O—C(═O)O, S(O)ₘ, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen and C₁-C₈alkyl; and
      Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl.

In certain aspects, compounds as described above are non-competitive CB1 antagonists.

The present invention further provides methods for treating a condition responsive to CB1 modulation in a patient, comprising administering to the patient a therapeutically effective amount of at least one compound as described herein. Such conditions include, for example, appetite disorders, obesity, dependency disorders such as alcohol dependency and nicotine dependency, asthma, liver cirrhosis, sepsis, irritable bowel disease, Crohn's disease, depression, schizophrenia, memory disorders, cognitive disorders, movement disorders and bone loss.

In further aspects, methods are provided for suppressing appetite in a patient, comprising administering to the patient an appetite reducing amount of at least one compound as described herein.

The present invention further provides pharmaceutical compositions, comprising (a) a first agent that is a compound of Formula I (or other formula provided herein), (b) a second agent that is suitable for treating one or more conditions responsive to CB1 modulation (e.g., an appetite disorder, obesity, an addictive disorder, asthma, liver cirrhosis, sepsis, irritable bowel disease, Crohn's disease, depression, schizophrenia, a memory disorder, a cognitive disorder, a movement disorder or bone loss); and (c) a physiologically acceptable carrier or excipient.

The present invention also provides packaged pharmaceutical preparations, comprising: (a) a composition comprising a compound of Formula I (or other formula provided herein) in a container; and (b) instructions for using the composition to treat one or more conditions responsive to CB1 modulation.

Methods are further provided herein for identifying a non-competitive CB1 antagonist, comprising: (a) contacting CB1 with a labeled non-competitive CB1 antagonist of Formula I (or other formula provided herein) and a test compound under conditions that permit binding of the labeled compound to CB1; (b) removing unbound labeled non-competitive CB1 antagonist and unbound test compound; (c) detecting a signal that corresponds to the amount of labeled non-competitive CB1 antagonist bound to the CB1; and (d) comparing the signal to a reference signal that corresponds to the amount of labeled non-competitive CB1 antagonist bound to the CB1 in the absence of test compound.

Within further aspects, the present invention provides methods for determining the presence or absence of CB1 in a sample, comprising: (a) contacting a sample with a compound as described herein under conditions that permit binding of the compound to CB1; and (b) detecting a level of the compound bound to CB1.

In yet another aspect, the invention provides methods of preparing the compounds disclosed herein, including the intermediates.

These and other aspects of the present invention will become apparent upon reference to the following detailed description.

### DETAILED DESCRIPTION

As noted above, the present invention provides CB1 antagonists. Such compounds may be used in vitro or in vivo in a variety of contexts as described herein.

Terminology

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. If a compound exists in various tautomeric forms, a recited compound is not limited to any one specific tautomer, but rather is intended to encompass all tautomeric forms. Certain compounds are described herein using a general formula that includes variables (e.g., Rₓ, Ar). Unless otherwise specified, each variable within such a formula is defined independently of any other variable, and any variable that occurs more than one time in a formula is defined independently at each occurrence.

A “pharmaceutically acceptable salt” of a compound is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

It will be apparent that each compound of Formula I (and other formulas provided herein) may, but need not, be formulated as a hydrate, solvate or non-covalent complex. In addition, the various crystal forms and polymorphs are within the scope of the present invention. Also provided herein are prodrugs of the compounds of Formula I and other formulas provided herein. A “prodrug” is a compound that may not fully satisfy the structural requirements of a formula provided herein, but is modified in vivo, following administration to a patient, to produce a compound of Formula I or other formula provided herein. For example, a prodrug may be an acylated derivative of a compound as provided herein. Prodrugs include compounds wherein hydroxy, amine or sulthydryl groups are bonded to any group that, when administered to a mammalian subject, cleaves to form a free hydroxy, amino, or sulfhydryl group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

As used herein, the term “alkyl” refers to a straight or branched chain saturated aliphatic hydrocarbon. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl or 3-methylpentyl. “C₀-C₄alkyl” refers to a single covalent bond (C₀) or an alkyl group having 1, 2, 3 or 4 carbon atoms; “C₀-C₆alkyl” refers to a single covalent bond or a C₁-C₆alkyl group; “C₀-C₈alkyl” refers to a single covalent bond or a C₁-C₈alkyl group.

“Alkylene” refers to a divalent alkyl group, as defined above. C₀-C₃alkylene is a single covalent bond or an alkylene group having 1, 2 or 3 carbon atoms.

“Alkenyl” refers to straight or branched chain alkene groups, in which at least one unsaturated carbon-carbon double bond is present. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

A “cycloalkyl” is a saturated or partially saturated cyclic group in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Certain cycloalkyl groups are C₃-C₈cycloalkyl, in which the ring contains from 3 to 8 ring members, all of which are carbon. A “(C₃-C₈cycloalkyl)C₀-C₄alkyl” is a C₃-C₈cycloalkyl group linked via a single covalent bond or a C₁-C₄alkylene group.

By “alkoxy,” as used herein, is meant an alkyl group attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are specific alkoxy groups.

Similarly, “alkylthio” refers to an alkyl group attached via a sulfur bridge.

The term “alkanoyl” refers to an acyl group in a linear or branched arrangement (e.g., —(C═O)-alkyl), where attachment is through the carbon of the keto group. Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁alkanoyl” refers to —(C═O)—H, which (along with C₂-C₈alkanoyl) is encompassed by the term “C₁-C₈alkanoyl.” Ethanoyl is C₂alkanoyl.

An “alkanone” is a ketone group in which carbon atoms are in a linear or branched alkyl arrangement. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone group has the structure —CH₂—(C═O)—CH₃.

Similarly, “alkyl ether” refers to a linear or branched ether substituent. Alkyl ether groups include C₂-C₈alkyl ether, C₂-C₆alkyl ether and C₂-C₄alkyl ether groups, which have 2 to 8, 6 or 4 carbon atoms, respectively. By way of example, a C₂ alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₁-C₈, C₁-C₆ and C₁-C₄alkoxycarbonyl groups, which have from 1 to 8, 6 or 4 carbon atoms, respectively, in the alkyl portion of the group. For example, “C₁alkoxycarbonyl” refers to —C(═O)—O—CH₃.

“Alkanoyloxy,” as used herein, refers to an alkanoyl group linked via an oxygen bridge (i.e., a group having the general structure —O—C(═O)-alkyl). Alkanoyloxy groups include C₂-C₈, C₂-C₆ and C₂-C₄alkanoyloxy groups, which have from 2 to 8, 6 or 4 carbon atoms, respectively. For example, “C₂alkanoyloxy” refers to —O—C(═O)—CH₃.

“Alkylsulfonyl” refers to groups of the formula —(SO₂)-alkyl, in which the sulfur atom is the point of attachment. Alkylsulfonyl groups include C₁-C₆alkylsulfonyl and C₁-C₄alkylsulfonyl groups, which have from 1 to 6 or 1 to 4 carbon atoms, respectively. “C₁-C₄haloalkylsulfonyl” is an alkylsulfonyl group of from 1 to 4 carbon atoms that is substituted with at least one halogen (e.g., trifluoromethylsulfonyl).

“Alkylamino” refers to a secondary or tertiary amine having the general structure —NH-alkyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups.

“Alkylaminoalkyl” refers to an alkylamino group linked via an alkylene group (i.e., a group having the general structure -alkyl-NH-alkyl or -alkyl-N(alkyl)(alkyl)) in which each alkyl is selected independently. Such groups include, for example, mono- and di-(C₁-C₈alkyl)aminoC₁-C₈alkyl, mono- and di-(C₁-C₆alkyl)aminoC₁-C₆alkyl and mono- and di-(C₁-C₆alkyl)aminoC₁-C₄alkyl, in which each alkyl may be the same or different. “Mono- or di-(C₁-C₆alkyl)aminoC₀-C₄alkyl” refers to a mono- or di-(C₁-C₆alkyl)amino group linked via a single covalent bond or a C₁-C₄alkylene group. The following are representative alkylaminoalkyl groups:

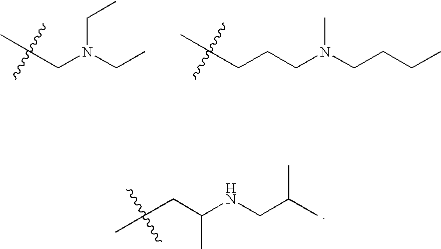

The term “aminocarbonyl” refers to an amide group (i.e., —C(═O)NH₂).

The term “aminosulfonyl” refers to a sulfonamide group (i.e., —SO₂NH₂).

The term “halogen” refers to fluorine, chlorine, bromine or iodine.

A “haloalkyl” is an alkyl group that is substituted with 1 or more halogen atoms (e.g., “C₁-C₈haloalkyl” groups have from 1 to 8 carbon atoms; “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “C₁-C₈haloalkoxy” groups have 1 to 8 carbon atoms.

A dash (“−”) that is not between two letters or numbers is used to indicate a point of attachment for a substituent. For example, —C(═O)NH₂ is attached through the carbon atom.

A “carbocycle” has from 1 to 3 fused, pendant or spiro rings, each of which has only carbon ring members. Typically, a carbocycle that has a single ring contains from 3 to 8 ring members (i.e., C₃-C₈carbocycles); rings having from 4 or 5 to 7 ring members (i.e., C₄-C₇carbocycles or C₅-C₇carbocycles) are recited in certain embodiments. Carbocycles comprising fused, pendant or spiro rings typically contain from 9 to 14 ring members. Carbocycles may be optionally substituted with a variety of substituents, as indicated. Unless otherwise specified, a carbocycle may be a cycloalkyl group (i.e., each ring is saturated or partially saturated as described above) or an aryl group (i.e., at least one ring within the group is aromatic). Representative aromatic carbocycles are phenyl, naphthyl and biphenyl. In certain embodiments preferred carbocycles have a single ring, such as phenyl and C₃-C₈cycloalkyl groups.

A “heterocycle” (also referred to herein as a “heterocyclic group”) has from 1 to 3 fused, pendant or spiro rings, at least one of which is a heterocyclic ring (i.e., one or more ring atoms is a heteroatom independently chosen from oxygen, sulfur and nitrogen, with the remaining ring atoms being carbon). Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and heterocycles comprising fused, pendant or spiro rings typically contain from 9 to 14 ring members. Certain heterocycles comprise a sulfur atom as a ring member; in certain embodiments, the sulfur atom is oxidized to SO or SO₂. Heterocycles may be optionally substituted with a variety of substituents, as indicated. Certain heterocycles are heteroaryl groups (i.e., at least one heterocyclic ring within the group is aromatic), such as a 5- to 10-membered heteroaryl (which may be monocyclic or bicyclic) or a 6-membered heteroaryl (e.g., pyridyl or pyrimidyl). Other heterocycles are heterocycloalkyl groups (i.e., no ring is aromatic).

A “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution.

Groups that are “optionally substituted” are unsubstituted or are substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

“CB1,” as used herein, refers to the human cannabinoid receptor reported by Hoeche et al. (1991) New Biol. 3(9):880-85, as well as allelic variants thereof and homologues thereof found in other species.

A “CB1 antagonist” is a compound that detectably inhibits signal transduction mediated by CB1. Such inhibition may be determined using the representative agonist-induced GTP binding assay provided in Example 8. Preferred CB1 antagonists have an IC₅₀ of 2 μM or less in this assay, more preferably 1 μM or less, and still more preferably 100 nM or less. In certain embodiments, the CB1 antagonist is specific for CB1 (i.e., the IC₅₀ value in a similar assay performed using the predominantly peripheral cannabinoid receptor CB2 is greater than 2 μM and/or the IC₅₀ ratio (CB2/CB1) is at least 10, preferably 100, and more preferably at least 1000). CB1 antagonists preferably have minimal agonist activity (i.e., induce an increase in the basal activity of CB1 that is less than 5% of the increase that would be induced by one EC₅₀ of the agonist CP55,940, and more preferably have no detectable agonist activity within the assay described in Example 8). CB1 antagonists for use as described herein are generally non-toxic.

A “non-competitive CB1 antagonist” is a CB1 antagonist that (1) does not detectably inhibit binding of CB1 agonist (e.g., CP55,940) to CB1 at antagonist concentrations up to 10 μM and (2) reduces the maximal functional response elicited by agonist. Compounds that satisfy these two conditions may be identified using the assays provided herein. Such compounds generally do not display detectable activity in the competition binding assay described in Example 7. In functional assays, a non-competitive antagonist, concentration-dependently reduces the maximal functional response elicited by agonist without altering agonist EC₅₀. The suppression of functional activity by a non-competitive antagonist cannot be overcome by increasing agonist concentrations (i.e., the antagonist activity is insurmountable).

A “therapeutically effective amount” (or dose) is an amount that, upon administration to a patient, results in a discernible patient benefit (e.g., provides detectable relief from a condition being treated). Such relief may be detected using any appropriate criteria, including the alleviation of one or more symptoms of dependency or an appetite disorder, or promotion of weight loss. In the case of appetite suppression, a therapeutically effect amount is sufficient to decrease patient appetite, as assessed using patient reporting or actual food intake. Such an amount is referred to herein as an “appetite reducing amount.” A therapeutically effective amount or dose generally results in a concentration of compound in a body fluid (such as blood, plasma, serum, CSF, synovial fluid, lymph, cellular interstitial fluid, tears or urine) that is sufficient to result in detectable alteration in CB1-mediated signal transduction (using an assay provided herein). The discernible patient benefit may be apparent after administration of a single dose, or may become apparent following repeated administration of the therapeutically effective dose according to a prescribed regimen, depending upon the indication for which the compound is administered.

A “patient” is any individual treated with a compound as provided herein. Patients include humans, as well as other animals such as companion animals (e.g., dogs and cats) and livestock. Patients may be experiencing one or more symptoms of a condition responsive to CB1 modulation or may be free of such symptom(s) (i.e., treatment may be prophylactic in a patient considered to be at risk for the development of such symptoms).

CB1 Antagonists

As noted above, the present invention provides certain arylalkyl ureas that may be used in a variety of contexts, including in the treatment of appetite disorders, obesity and addictive disorders. Such compounds may also be used within in vitro assays (e.g., assays for CB1 activity), as probes for detection and localization of CB1 and within assays to identify other CB1 antagonists, including non-competitive CB1 antagonists.

Compounds provided herein generally satisfy Formula I. Within certain such compounds, Ar₁ is phenyl, pyridyl or pyrimidyl, each of which is substituted with from 0 to 3 substituents independently located meta or para to the point of attachment. In other words, if Ar₁ is phenyl, then Ar₁ is unsubstituted or substituted at the 3, 4 and/or 5 position. Similarly, if Ar₁ is pyridin-2-yl, the pyridin-2-yl is either unsubstituted or substituted at the 4, 5 and/or 6 position. The pyrimidinyl group 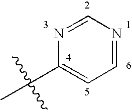
is preferably substituted at the 2 and/or 6 position, and 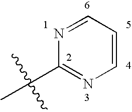
is preferably substituted at the 4, 5 and/or 6 position. Preferably, each substituent is independently: (a) halogen, hydroxy or cyano; or (b) C₁-C₄alkyl, C₁-C₄alkoxy, C₁-C₄alkylthio, C₁-C₄haloalkyl, C₁-C₄haloalkoxy, C₁-C₄alkanoyl, C₁-C₄alkanoyloxy or C₁-C₄alkoxycarbonyl, each of which is substituted with from 0 to 2 substituents independently chosen from hydroxy, halogen, C₁-C₄alkyl, C₁-C₄alkoxy, C₂-C₄alkanoyl and C₁-C₄haloalkyl.

Ar₁, within certain such compounds, is phenyl that is unsubstituted or substituted with 1 or 2 substituents, each of which is located meta or para to the point of attachment, and each of which is independently halogen, cyano, nitro, C₁-C₄alkyl, C₁-C₄alkoxy, C₁-C₄alkylthio, C₁-C₄haloalkyl or C₁-C₄haloalkoxy. Representative such Ar₁ groups include, for example, phenyl, 3-difluoromethoxy-phenyl, 4-difluoromethoxy-phenyl, 3-(C₁-C₄alkyl)phenyl, 4-(C₁-C₄alkyl)phenyl, 3-(C₁-C₄alkoxy)phenyl and 4-(C₁-C₄alkoxy)phenyl.

R, within certain compounds, is phenylC₁-C₄alkyl that is substituted with from 0 to 3 substitutents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy. Within other compounds, R is (C₃-C₈cycloalkyl)C₀-C₄alkyl or (3- to 8-membered heterocycloalkyl)C₀-C₄alkyl, each of which is substituted with from 0 to 3 substitutents independently chosen from halogen, C₁-C₆alkyl, (C₃-C₈cycloalkyl)C₀-C₄alkyl, C₁-C₄alkoxy, C₁-C₄alkoxycarbonyl, C₁-C₂haloalkoxy, phenylC₀-C₄alkyl and (6-membered heteroaryl)C₀-C₄alkyl. In still further compounds, R is (C₃-C₈cycloalkyl)C₀-C₄alkyl or (3- to 8-membered heterocycloalkyl)C₀-C₄alkyl, each of which is fused to a phenyl or 6-membered heteroaryl ring and substituted with from 0 to 3 substitutents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

Certain compounds of Formula I further satisfy Formula Ia: 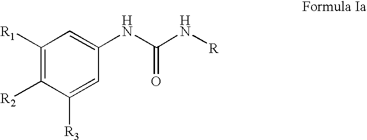
wherein R₁, R₂ and R₃ are independently chosen from hydrogen and R_{y}, and wherein at least one of R₁ and R₂ is not hydrogen. In certain such compounds, R₁, R₂ and R₃ are independently chosen from hydrogen, halogen, cyano, C₁-C₄alkyl, C₁-C₄haloalkyl, C₁-C₄alkoxy, C₁-C₄haloalkoxy, C₁-C₄alkanoyloxy and C₁-C₄alkoxycarbonyl.

Still further compounds of Formula I or Ia further satisfy Formula II: 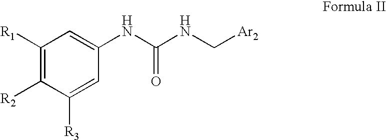
wherein Ar₂ is phenyl or a 6-membered heteroaryl, each of which is substituted with from 1 to 3 substitutents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl and C₁-C₂haloalkoxy.

Other compounds of Formula I of Formula Ia further satisfy Formula III: 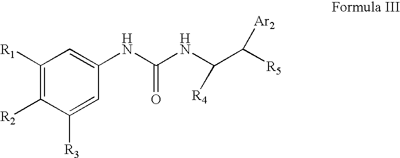
wherein:
R₄ and R₅ are independently chosen from hydrogen, C₁-C₆alkyl and (C₃-C₈cycloalkyl)C₀-C₄alkyl; or R₄ and R₅ are taken together to form a C₃-C₆cycloalkyl group; and
Ar₂ is phenyl or a 6-membered heteroaryl, each of which is substituted with from 0 to 3 substitutents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl, C₁-C₂haloalkyl and C₁-C₂haloalkoxy.

Certain compounds of Formula III further satisfy Formula IIIa: 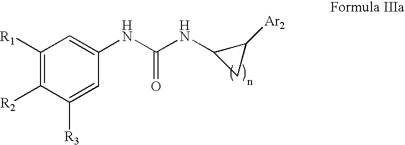
wherein n is 1, 2, 3 or 4.

Other compounds of Formula III further satisfy Formula IIIb: 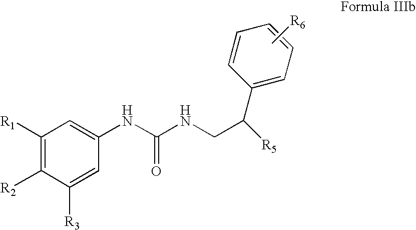
wherein:
R₅ is hydrogen, C₁-C₆alkyl or (C₃-C₈cycloalkyl)C₀-C₄alkyl; and
R₆ represents from 0 to 3 substituents independently chosen from hydroxy, halogen, C₁-C₆alkyl, C₁-C₄alkoxy, C₁-C₂haloalkyl and C₁-C₂haloalkoxy.

Certain compounds of Formula I or Formula Ia further satisfy Formula IV: 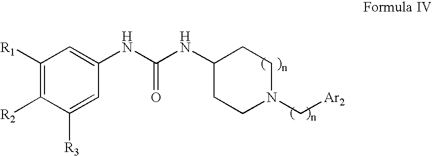
wherein:
Ar₂ is phenyl or a 6-membered heteroaryl, each of which is substituted with from 0 to 3 substitutents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl, C₁-C₂haloalkyl and C₁-C₂haloalkoxy; and
each n is independently 0 or 1.

Certain compounds of Formula I or Formula Ia further satisfy Formula V: 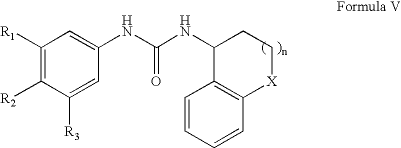
wherein X is CH, N or O; and n is 0, 1 or 2.

Representative compounds provided herein include, but are not limited to, those specifically described in the Examples below. It will be apparent that the specific compounds recited herein are representative only, and are not intended to limit the scope of the present invention. Further, as noted above, all compounds of the present invention may be present as a free acid or base or as a pharmaceutically acceptable salt.

As noted above, compounds provided herein are CB1 antagonists. Certain such compounds are non-competitive CB1 antagonists. In addition, or alternatively, certain compounds provided herein display CB1 specificity. CB1 antagonist activity may be confirmed using an agonist-induced GTP binding assay, such as the assay described in Example 8. Such assays employ a CB1-containing cell membrane preparation (e.g., a preparation of membranes of insect cells that recombinantly express CB1) to determine the effect of a test compound on CB1 agonist-induced GTP binding to CB1. Briefly, a first cell membrane preparation comprising CB1 is contacted with: (i) labeled GTP; (ii) a CB1 agonist; and (iii) a test compound to yield a test membrane preparation. Simultaneously, or in either order, a second cell membrane preparation comprising CB1 is contacted with: (i) labeled GTP; and (ii) a CB1 agonist to yield a control membrane preparation. The labeled GTP is preferably GTPγ³⁵S; a representative CB1 agonist is CP55,940. Such contact is performed under conditions that are suitable for GTP binding to CB1, such as the conditions described in Example 8. The concentrations of labeled GTP and CB1 agonist used are generally concentrations that are sufficient to result in a detectable increase in the amount of labeled GTP bound to the membrane preparation in the presence of CB1 agonist. Such concentrations may be determined by routine experimentation; representative suitable concentrations are provided in Example 8. Generally, a range of test compound concentrations is used (e.g., ranging from 10⁻¹⁰M to 10⁻⁵M).

After sufficient contact (e.g., incubation) to allow GTP binding to the membrane preparations, a signal that corresponds to (represents) the amount of bound, labeled GTP is detected (typically, unbound labeled GTP is first removed via a washing step). In other words, simultaneously or in either order: (i) a test signal that represents an amount of bound, labeled GTP in the test membrane preparation is detected; and (ii) a control signal that represents an amount of bound, labeled GTP in the control membrane preparation is detected. The nature of the signal detected is determined by the type of label used. For example, if the GTP is radioactively labeled, the signal detected is radioactive decay (e.g., via liquid scintillation spectrometry). The CB1 antagonist activity of the test compound is then determined by comparing the test signal with the control signal. A test signal that is lower than the control signal indicates that the test compound is a CB1 antagonist.

In certain embodiments, preferred compounds are cannabinoid receptor-specific. This means that they only bind to, activate, or inhibit the activity of certain receptors other than cannabinoid receptors (preferably other than CB1) with affinity constants of greater than 100 nanomolar, preferably greater than 1 micromolar, more preferably greater than 4 micromolar. Alternatively, or in addition, such compounds exhibit 200-fold greater affinity for CB1 than for other non-cannabinoid cellular receptors. Such other non-cannabinoid cellular receptors include histamine receptors, bioactive peptide receptors (including NPY receptors such as NPY Y5), and hormone receptors (e.g., melanin-concentrating hormone receptors). Assays for evaluating binding to such receptors are well known, and include those disclosed in U.S. Pat. No. 6,566,367, which is incorporated herein by reference for its disclosure of NPY receptor binding assays in Example 676 columns 82-83; and in PCT International Application Publication No. WO 02/094799 which is incorporated herein by reference for its disclosure of an MCH receptor binding assay in Example 2, pages 108-109.

The usefulness of the compounds provided herein for the various diseases and disorders may be demonstrated in animal disease models that are known in the art, such as (but not limited to):
Colombo et al. (1998) Life Sciences 63:113-17 and Vickers and Kennett (2005) Curr. Drug. Targets 6:215-23—food intake and weight loss (rats)
Simiand et al. (1998) Behavioral Pharm. 9:179-181—sweet food intake (marmosets)
Rowland et al. (2001) Psychopharm. 159:111-16—food intake (rats)
Amone et al. (1997) Psychopharm. 132:104-106—sucrose and ethanol intake (mice)
Colombo et al. (2004) Eur. J. Pharmacol. 498:119-23—alcohol motivational properties (rats)
Serra et al. (2002) Eur. J. Pharmacol. 443:95-97—alcohol deprivation effect (rats)
Rubino et al. (2000) Life Sciences 22:2213-29—opiate withdrawal syndrome (rats)
Chaperon et al. (1998) Psychopharm. 135:324-32—motor activity, place conditioning (rats)
Abraham et al. (1993) J. Clin. Invest. 93:776 and Milne and Piper (1995) Eur. J. Pharmacol. 282:243—bronchial hyperresponsiveness (sheep and guinea pigs)
Kadoi et al. (2005) British Journal of Anaesthesia 94(5):563-68—septic shock (rats)
Batkai et al. (2001) Nature Medicine 7(7):827-32-vasodilation in liver cirrhosis (rats)
Tsusumi et al. (2000) Biol. Pharm. Bull. (Japan) 23(5):657-59—constipation (monkeys)
Kapur (2001) J. Pathology 194(3):277-88—chronic intestinal pseudo-obstruction (rodents)

If desired, compounds provided herein may be evaluated for certain pharmacological properties including, but not limited to, oral bioavailability (preferred compounds are orally bioavailable to an extent allowing for therapeutically effective doses of less than 140 mg/kg, preferably less than 50 mg/kg, more preferably less than 30 mg/kg, even more preferably less than 10 mg/kg, still more preferably less than 1 mg/kg and most preferably less than 0.1 mg/kg), toxicity (a preferred compound is nontoxic when a therapeutically effective amount is administered to a subject), side effects (a preferred compound produces side effects comparable to placebo when a therapeutically effective amount of the compound is administered to a subject), serum protein binding and in vitro and in vivo half-life (a preferred compound exhibits an in vivo half-life allowing for Q.I.D. dosing, preferably T.I.D. dosing, more preferably B.I.D. dosing, and most preferably once-a-day dosing). In addition, differential penetration of the blood brain barrier may be desirable. Routine assays that are well known in the art may be used to assess these properties, and identify superior compounds for a particular use. For example, assays used to predict bioavailability include transport across human intestinal cell monolayers, including Caco-2 cell monolayers. Penetration of the blood brain barrier of a compound in humans may be predicted from the brain levels of the compound in laboratory animals given the compound (e.g., intravenously). Serum protein binding may be predicted from albumin binding assays. Compound half-life is inversely proportional to the frequency of dosage of a compound. In vitro half-lives of compounds may be predicted from assays of microsomal half-life as described herein.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, or (4) does not cause substantial release of liver enzymes.

As used herein, a compound that does not substantially inhibit cellular ATP production is a compound that satisfies the criteria set forth in Example 10, herein. In other words, cells treated as described in Example 10 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more highly preferred embodiments, such doses do not elevate such serum levels by more than 75% or 50% over matched controls. Alternatively, a compound does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. For example, such compounds may have one or more atoms replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, for example increased in vivo half-life or reduced dosage requirements and, hence, may be preferred in some circumstances.

Preparation of CB1 Antagonists

Compounds provided herein may generally be prepared using standard synthetic methods. In general, starting materials are commercially available from suppliers such as Sigma-Aldrich Corp. (St. Louis, Mo.), or may be synthesized from commercially available precursors using established protocols. By way of example, a synthetic route similar to that shown in any of the following Schemes may be used, together with synthetic methods known in the art of synthetic organic chemistry, or variations thereon as appreciated by those skilled in the art. Those skilled in the art will recognize that the reagents and synthetic transformations in the following Schemes can be readily modified to produce additional compounds of Formula I. Each variable in the following Schemes refers to any group consistent with the description of the compounds provided herein.

When a protecting group is required, an optional deprotection step may be employed. Suitable protecting groups and methodology for protection and deprotection such as those described in Protecting Groups in Organic Synthesis by T. Greene are well known. Compounds and intermediates requiring protection/deprotection will be readily apparent.

Certain definitions used in the following Schemes and in the Examples include:
CDCl₃ deuterated chloroform
DIEA diiosopropylethylamine
DPPA diphenylphosphoryl azide
EtOAc ethyl acetate
eq. equivalent(s)
¹H NMR proton nuclear magnetic resonance
Hz hertz
LC/MS liquid chromatography/mass spectrometry
MHz megahertz
MS mass spectrometry
(M+1) mass+1
δ chemical shift
Ph phenyl
Pd(PPh₃)₄ tetrakis(triphenylphosphine) palladium (0)

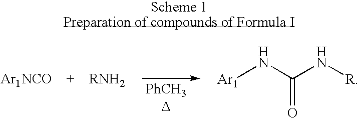

Scheme 1 illustrates a method for preparing compounds of Formula I from readily available aminoaryl compounds and arylisocyanates. In this method, on equivalent of arylisocyanate is heated an alkylamine derivative in an appropriate solvent. Commonly used solvents for the reaction include but are not limited to toluene, tetrahydrofuran and dioxane. Those skilled in the art will recognize that the choice of solvent and reaction temperature may be modified to optimize the reaction for various reactant combinations.

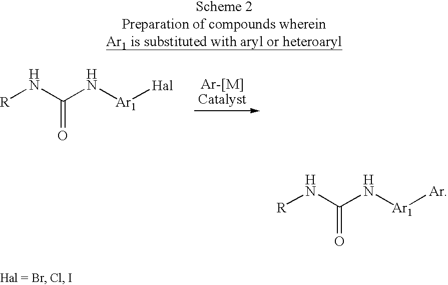

Scheme 2 illustrates a method for preparing compounds of Formula I wherein Ar₁ is substituted with an aryl or heteraryl group (Ar). In Scheme 2, the haloaryl urea is coupled to various aryl groups via a transition metal-catalyzed coupling reaction with a metalloaryl reagent (Ar-[M]). Suitable reagent/catalyst pairs include aryl boronic acid/palladium(0) (Suzuki reaction; Miyaura and Suzuki (1995) Chemical Review 95:2457) and aryl trialkylstannane/palladium(0) (Stille reaction; Mitchell, Synthesis 1992:803). Palladium(0) represents a catalytic system made of a combination of metal/ligand pair such as, but not limited to, tetrakis(triphenylphosphine)palladium(0), palladium(II) acetate/tri(o-tolyl)phosphine, tris(dibenzylideneacetone)dipalladium(0)/tri-tert-butylphosphine or dichloro[1,1′-bis(diphenylphosphine)ferrocene]palladium(0). Nickel(II) represents a nickel-containing catalyst such as [1,2-bis(diphenylphosphino)ethane]dichloronickel(II) or [1,3-bis(diphenylphosphino)propane]dichloronickel(II).

In certain embodiments, a compound provided herein may contain one or more asymmetric carbon atoms, so that the compound can exist in different stereoisomeric forms. Such forms can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example a chiral HPLC column.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

Pharmaceutical Compositions

The present invention also provides pharmaceutical compositions comprising one or more compounds provided herein, together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. In addition, other active ingredients may (but need not) be included in the pharmaceutical compositions provided herein.

Pharmaceutical compositions may be formulated for any appropriate manner of administration, including, for example, inhalation (e.g., nasal or oral) or topical, oral, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions suitable for oral use are preferred. Such compositions include, for example, tablets, troches, lozenges, aqueous or oily suspensions, dispersible powders or granules, emulsion, hard or soft capsules, or syrups or elixirs. Within yet other embodiments, pharmaceutical compositions may be formulated as a lyophilizate. Formulation for topical administration may be preferred for certain conditions (e.g., in the treatment of skin conditions such as burns or itch). Formulation for direct administration into the bladder (intravesicular administration) may be preferred for treatment of urinary incontinence and overactive bladder. Formulations for inhalation include powders and liquids suitable for inhalation or insufflation, and solutions and suspensions suitable for nebulization.

Compositions intended for oral use may further comprise one or more components such as sweetening agents, flavoring agents, coloring agents and/or preserving agents in order to provide appealing and palatable preparations. Tablets contain the active ingredient in admixture with physiologically acceptable excipients that are suitable for the manufacture of tablets. Such excipients include, for example, inert diluents to increase the bulk weight of the material to be tableted (e.g., calcium carbonate, sodium carbonate, lactose, calcium phosphate and sodium phosphate), granulating and disintegrating agents that modify the distintegration rate in the environment of use (e.g., corn starch, starch derivatives, alginic acid and salts of carboxymethylcellulose), binding agents that impart cohesive qualities to the powdered material(s) (e.g., starch, gelatin, acacia and sugars such as sucrose, glucose, dextrose and lactose) and lubricating agents (e.g., magnesium stearate, calcium stearate, stearic acid and talc). Tablets may be formed using standard techniques, including dry granulation, direct compression and wet granulation. The tablets may be uncoated or they may be coated by known techniques.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions contain the active material(s) in admixture with one or more suitable excipients, such as suspending agents (e.g., sodium carboxymethylcellulose, methylcellulose, hydroxypropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, such as ethyl or n-propyl p-hydroxybenzoate, one or more coloring agents, one or more flavoring agents, and/or one or more sweetening agents, such as sucrose or saccharin.

Oily suspensions may be formulated by suspending the active ingredient(s) in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified above. Additional excipients, such as sweetening, flavoring and coloring agents, may also be present.

Pharmaceutical compositions may also be formulated as oil-in-water emulsions. The oily phase may be a vegetable oil (e.g., olive oil or arachis oil), a mineral oil (e.g., liquid paraffin) or a mixture thereof. Suitable emulsifying agents include naturally-occurring gums (e.g., gum acacia or gum tragacanth), naturally-occurring phosphatides (e.g., soy bean lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Remington: The Science and Practice of Pharmacy, 21^{st} ed., Lippincott Williams & Wilkins, Philadelphia, Pa. (2005). Formulations may comprise microcapsules, such as hydroxymethylcellulose or gelatin-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids and emulsions. Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used.

A pharmaceutical composition may be prepared as a sterile injectible aqueous or oleaginous suspension. The compound(s) provided herein, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Pharmaceutical compositions may also be formulated as suppositories (e.g., for urethral or rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols. Suppositories include those described by, for example, U.S. Pat. No. 6,846,823, which is hereby incorporated by reference for its teaching of the preparation and administration of pharmaceutical compositions for urethral or rectal administration. Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated as controlled release formulations (i.e., a formulation such as a capsule, tablet or coated tablet that slows and/or delays release of active ingredient(s) following administration), which may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, diputyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed by methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 5,524,060; 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217; each of which is hereby incorporated by reference for its teaching of the preparation of controlled release dosage forms.

In addition to or together with the above modes of administration, a compound provided herein may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Compound(s) provided herein are generally administered in a therapeutically effective amount. Preferred systemic doses are no higher than 50 mg per kilogram of body weight per day (e.g., ranging from about 0.001 mg to about 50 mg per kilogram of body weight per day), with oral doses generally being about 5-20 fold higher than intravenous doses (e.g., ranging from 0.01 to 40 mg per kilogram of body weight per day).

The amount of active ingredient that may be combined with the carrier materials to produce a single dosage unit will vary depending, for example, upon the patient being treated and the particular mode of administration. Dosage units will generally contain between from about 10 μg to about 500 mg of an active ingredient. In certain embodiments, the dosage unit contains an amount of the compound that is sufficient to effect a decrease in the patient's caloric intake (i.e., an appetite-suppressing amount). Optimal dosages may be established using routine testing, and procedures that are well known in the art.

Pharmaceutical compositions may be used for treating a condition responsive to CB1 modulation. Such conditions include, for example:
appetite disorders (e.g., binge eating disorder, bulimia, anorexia);
obesity;
weight loss or control (e.g., reducing calorie or food intake and/or appetite suppression);
addictive disorders such as:
   alcohol dependency (e.g., alcohol abuse, addiction and/or dependency including treatment for abstinence, craving reduction and relapse prevention of alcohol intake);
   nicotine dependency (e.g., smoking addiction, cessation and/or dependency including treatment for craving reduction and relapse prevention of tobacco smoking); and
   drug dependency (e.g., chronic treatment with or abuse of drugs such as opioids, barbiturates, cannabis, cocaine, amphetamines, phencyclide, hallucinogens, and/or benzodiazepines); and
bone loss (e.g., resulting from estrogen deficiency).

Other conditions responsive to CB1 modulation include CNS disorders (e.g., anxiety, depression, panic disorder, bipolar disorder, psychosis, schizophrenia, behavioral addiction, dementia (including memory loss, Alzheimer's disease, dementia of aging, vascular dementia, mild cognitive impairment, age-related cognitive decline, and mild neurocognitive disorder), attention deficit disorder (ADD/ADHD), amnesia, cognitive disorders, memory disorders, neurodegeneration, cerebellar and spinocerebellar disorder, cranial trauma, obsessive-compulsive disorder, senile dementia, impulsivity), thymic disorders, Tourette's syndrome, Huntington's chorea, Raynaud's syndrome, peripheral neuropathy, diabetes (type II or non insulin dependent), glaucoma, migraine, seizure disorders, epilepsy, locomotor disorders (movement disorders induced by medicaments, dyskinesias or Parkinson's disease), respiratory disorders (such as asthma), gastrointestinal disorders (e.g., dysfunction of gastrointestinal motility or intestinal propulsion, irritable bowel syndrome, Crohn's disease), liver cirrhosis, vomiting, diarrhea, ulcer, multiple sclerosis, cardiovascular disorder, dystonia, endotoxemic shocks, hemorrhagic shocks, hypotension, insomnia, a disorder of the endocrine system, urinary or bladder disorders, cancer, infectious disease, inflammation, infection, cancer, neuroinflammation (such as atherosclerosis), Guillain-Barre syndrome, viral encephalitis, cranial trauma, sepsis or a reproductive disorder. In certain embodiments, the agent is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder or bone loss.

Certain pharmaceutical compositions provided herein comprise a first agent that is a compound of Formula I in combination with a second agent that differs in structure from the first agent and is suitable for treating the condition of interest. In certain embodiments, the second agent is not a compound of Formula I; in further embodiments, the second agent is not a CB1 antagonist. In certain such compositions, the second agent is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder and/or bone loss. Representative second agents for use within such pharmaceutical compositions include anti-obesity agents such as MCH receptor antagonists, apo-B/MTP inhibitors, 11β-hydroxy steroid dehydrogenase-1 inhibitors, peptide YY₃-36 or an analog thereof, MCR-4 agonists, CCK-A agonists, monoamine reuptake inhibitors, sympathomimetic agents, β₃ adrenergic receptor agonists, dopamine agonists, melanocyte-stimulating hormone receptor analogues, 5-HT2c receptor agonists, leptin or an analog thereof, leptin receptor agonists, galanin antagonists, lipase inhibitors, bombesin agonists, neuropeptide-Y receptor antagonists, thyromimetic agents, dehydroepiandrosterone or analog thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists, and neuromedin U receptor agonists. Such agents include, for example, phentermine, orlistat and sibutramine (e.g., sibutramine HCl monohydrate, sold as Meridia® (Abbott Laboratories)).

Certain second agents for use in weight management are MCH receptor antagonists. Preferably, such MCH receptor antagonists detectably inhibit MCH binding to MCHR1 and/or MCHR2 (as determined using a standard in vitro MCH receptor ligand binding assay and/or calcium mobilization assay) at submicromolar concentrations, preferably at nanomolar concentrations, and more preferably at subnanomolar concentrations. In certain preferred embodiments, MCH receptor antagonists for use herein detectably inhibit MCH binding to MCHR1. Briefly, a competition assay is performed in which a MCH receptor preparation is incubated with labeled (e.g., ¹²⁵I) MCH and unlabeled test compound. The MCH receptor used is preferably a mammalian MCHR1 or MCHR2, more preferably a human or monkey MCHR1 or MCHR2. The MCH receptor preparation may be, for example, a membrane preparation from HEK293 cells that recombinantly express a human MCH receptor (e.g., Genbank Accession No. Z86090), monkey MCHR1 (such as the MCHR1 sequence provided in SEQ ID NO:1 of WO 03/060475), or human MCHR1/human beta-2-adrenergic chimeric receptor. Incubation with a MCH receptor antagonist results in a decrease in the amount of label bound to the MCH receptor preparation, relative to the amount of label bound in the absence of the antagonist. Preferably, a MCH receptor antagonist exhibits a Kᵢ at a MCH receptor of less than 1 micromolar, binding specifically and with high affinity to a MCH receptor. More preferably, such a compound exhibits a Kᵢ at a MCH receptor of less than 500 nM, 100 nM, 20 nM or 10 nM.

In certain embodiments, MCHR antagonists include substituted 1-benzyl-4-aryl piperazine and piperidine analogues, as described within pending U.S. patent application Ser. No. 10/152,189, which published as US 2005-0065162 on Mar. 24, 2005. The corresponding PCT application published as WO 02/094799 on Nov. 28, 2002, and this disclosure is hereby incorporated herein by reference for its teaching of MCHR antagonists (pages 3-5, 20-25 and 74-107) and the preparation thereof (pages 29-42 and 50-73). Within other embodiments, MCH receptor antagonists for use as described herein are substituted benzimidazole analogues as described within pending U.S. patent application Ser. No. 10/399,499, filed Jan. 9, 2003. The corresponding PCT application published as WO 03/060475 on Jul. 24, 2003, and this disclosure is hereby incorporated herein by reference for its teaching of MCH receptor antagonists (pages 2-5, Table I (pages 14-19) and Table II (pages 38-48) and the preparation thereof (pages 23-24 and 32-38). Within further embodiments, MCH receptor antagonists are as described within pending U.S. patent application Ser. No. 10/399,111, filed Jan. 9, 2003. The corresponding PCT application published as WO 03/059289 on Jul. 24, 2003, and this disclosure is hereby incorporated herein by reference for its teaching of MCH receptor antagonists (pages 3-4 and 31-50) and the preparation thereof (pages 19-20 and 28-31). Within further embodiments, MCH receptor antagonists include those described within U.S. Pat. No. 6,569,861, which is hereby incorporated by reference for its teaching of phenylcycloalkylmethylamino and phenylalkenylamino MCH receptor antagonists (columns 3-9 and 18-19) and the preparation thereof (columns 16-18). Still further MCH receptor antagonists are described, for example, within the following published PCT applications: WO 03/097047, WO 03/087046, WO 03/087045, WO 03/087044, WO 03/072780, WO 03/070244, WO 03/047568, WO 03/045920, WO 03/045918, WO 03/045313, WO 03/035055, WO 03/033480, WO 03/015769, WO 03/028641, WO 03/013574, WO 03/004027, WO 02/089729, WO 02/083134, WO 02/076947, WO 02/076929, WO 02/057233, WO 02/051809, WO 02/10146, WO 02/06245, WO 02/04433, WO 01/87834, WO 01/82925, WO 01/57070, WO 01/21577 and WO 01/21169, as well as Japanese Application Publication Number 2001-226269. It will be apparent that the above are illustrative examples of MCH receptor antagonists, and are not intended to limit the scope of the present invention.

Representative second agents suitable for treating an addictive disorder include, for example, Methadone, LAAM (levo-alpha-acetyl-methadol), naltrexone (e.g., ReVia™), ondansetron (e.g., Zofran®), sertraline (e.g., Zoloft®), fluoxetine (e.g., Prozac®), diazepam (e.g., Valium®) and chlordiazepoxide (e.g., Librium), varenicline and buproprion (e.g., Zyban® or Wellbutrin®). Other representative second agents for use within the pharmaceutical compositions provided herein include nicotine receptor partial agonists, opioid antagonists and/or dopaminergic agents.

Pharmaceutical compositions may be packaged for treating conditions responsive to CB1 modulation (e.g., treatment of appetite disorder, obesity and/or addictive disorder, or other disorder indicated above). Packaged pharmaceutical preparations generally comprise a container holding a therapeutically effective amount of a pharmaceutical composition as described above and instructions (e.g., labeling) indicating that the composition is to be used for treating a condition responsive to CB1 modulation in a patient. In certain embodiments, a packaged pharmaceutical preparation comprises one or more compounds provided herein and one or more additional agents in the same package, either in separate containers within the package or in the same container (i.e., as a mixture). Preferred mixtures are formulated for oral administration (e.g., as pills, capsules, tablets or the like). In certain embodiments, the package comprises a label bearing indicia indicating that the components are to be taken together for the treatment of an appetite disorder, obesity, an addictive disorder, asthma, liver cirrhosis, sepsis, irritable bowel disease, Crohn's disease, depression, schizophrenia, a memory disorder, a cognitive disorder, a movement disorder and/or bone loss.

Methods of Use

Within certain aspects, the present invention provides methods for treating a condition responsive to CB1 modulation in a patient. The patient may be afflicted with such a condition, or may be free of symptoms but considered at risk for developing such a condition. A condition is “responsive to CB1 modulation” if the condition or symptom(s) thereof are alleviated, attenuated, delayed or otherwise improved by modulation of CB1 activity. Such conditions include, for example, appetite disorders, obesity, addictive disorders, asthma, liver cirrhosis, sepsis, irritable bowel disease, Crohn's disease, depression, schizophrenia, memory disorders, cognitive disorders, movement disorders and bone loss, as well as other disorders indicated above. In general, such methods comprise administering to the patient a therapeutically effective amount of at least compound according to Formula I. In related aspects, the present invention provides methods for appetite suppression in a patient, comprising administering to a patient in need thereof a therapeutically effective amount of at least compound according to Formula I.

It will be apparent that compounds provided herein may be administered alone or in combination with one or more additional agents that are suitable for treating the condition of interest. Within combination therapy, the compound(s) of Formula I and additional agent(s) may be present in the same pharmaceutical composition, or may be administered separately in either order. Representative additional agents for use in such methods include the second agents described above.

Suitable dosages for compounds provided herein within such combination therapy are generally as described above. Dosages and methods of administration of the additional agent(s) can be found, for example, in the manufacturer's instructions or in the Physician's Desk Reference. In certain embodiments, combination administration results in a reduction of the dosage of the additional agent required to produce a therapeutic effect (i.e., a decrease in the minimum therapeutically effective amount). Thus, preferably, the dosage of additional agent in a combination or combination treatment method of the invention is less than the maximum dose advised by the manufacturer for administration of the agent without combination with a compound of Formula I. More preferably this dose is less than ¾, even more preferably less than ½, and highly preferably, less than ¼ of the maximum dose, while most preferably the dose is less than 10% of the maximum dose advised by the manufacturer for administration of the agent(s) when administered without combination administration as described herein. It will be apparent that the dose of compound of Formula I needed to achieve the desired effect may similarly be affected by the dose and potency of the additional agent.

Administration to the patient can be by any means discussed above, including oral, topical, nasal or transdermal administration, or intravenous, intramuscular, subcutaneous, intrathecal, epidural, intracerebroventrilcular or like injection. Oral administration is preferred in certain embodiments (e.g., formulated as pills, capsules, tablets or the like).

Treatment regimens may vary depending on the compound used and the particular condition to be treated. In general, a dosage regimen of 4 times daily or less is preferred, with 1 or 2 times daily particularly preferred. It will be understood, however, that the specific dose level and treatment regimen for any particular patient will depend upon a variety of factors including the activity of the specific compound employed, the age, body weight, general health, sex, diet, time of administration, route of administration, and rate of excretion, drug combination and the severity of the particular disease undergoing therapy. Dosages are generally as described above; in general, the use of the minimum dose sufficient to provide effective therapy is preferred. Patients may generally be monitored for therapeutic effectiveness using medical or veterinary criteria suitable for the condition being treated or prevented. For example, treatment of obesity is considered to be effective if it results in a statistically significant decrease in weight or BMI.

For combination therapy, pharmaceutical compositions may be formulated in single-dose units (e.g., tablets or capsules). Each unit may contain both the compound of Formula I and the second agent; alternatively, each unit may contain a single agent, with the units coadministered to achieve combination therapy. Within single-dose units, the compound of Formula I and second agent (e.g., MCH receptor antagonist) are present in therapeutically effective amounts (i.e., an amount that results in a discernible benefit in a patient when the compound of Formula I and second agent are administered contemporaneously and repeatedly at a prescribed frequency (e.g., from 1 to 4 times per day for a period of weeks or months) to a patient. Such benefit(s) include those described above, such as decreased BMI, decreased appetite or food intake and/or weight loss. A therapeutically effective amount of second agent is an amount that results in such a discernible patient benefit when so administered, as compared to the patient benefit observed following administration of compound of Formula I alone. Similarly, a therapeutically effective amount of compound of Formula I is an amount that results in such a discernible patient benefit when so administered, as compared to the patient benefit observed following administration of second agent alone. “Contemporaneously,” as used herein, refers to a time frame such that the second agent is present in a body fluid of a patient (at a therapeutic concentration) at the same time as the CB1 antagonist is present in the body fluid (at a therapeutic concentration). Contemporaneous administration is also referred to herein as “coadministration.”

It will be apparent that the therapeutically effective amount in the context of combination therapy may be lower than the therapeutically effective amount for an agent administered alone. In certain embodiments, a therapeutically effective amount of second agent is lower than the amount that would need to be administered to effect a comparable patient benefit in the absence of compound of Formula I. Within certain compositions and methods provided herein, at least an additive effect is observed (i.e., the patient benefit is at least the sum of the benefits that would be achieved by the separate administration of the same amounts of second agent and compound of Formula I).

In certain embodiments, the therapeutically effective amount of second agent is less than ¾, ½, ¼ or 10% of the maximum recommended dose for the MCHR antagonist (i.e., the maximum dose advised by the manufacturer or the U.S. Food and Drug Administration (FDA)). Similarly, in certain embodiments, a therapeutically effective amount of compound of Formula I is lower than the amount that would need to be administered to effect a comparable patient benefit in the absence of second agent. In certain embodiments, the therapeutically effective amount of compound of Formula I is less than ¾, ½, ¼ or 10% of the maximum recommended dose for the compound of Formula I (i.e., the maximum dose advised by the manufacturer or the FDA).

In further embodiments, the therapeutically effective amount of second agent is less than the minimum dose of the second agent proven effective in a United States clinical trial of the second agent, wherein the trial is conducted without coadministration of compound of Formula I (e.g., the therapeutically effective amount is less than 95%, less than 90%, less than 75% or less than 50% of the minimum dose proven effective in such a clinical trial). In other embodiments, the therapeutically effective amount of compound of Formula I is lower than the minimum dose of the compound proven effective in a United States clinical trial of the compound, wherein the trial is conducted without coadministration of a second agent (e.g., the therapeutically effective amount is less than 95%, less than 90%, less than 75% or less than 50% of the minimum dose proven effective in such a clinical trial). In still further such embodiments, both the second agent and the compound of Formula I are employed at doses that are lower than the minimum dose proven effective in such clinical trials. The phrase “clinical trial,” as used herein, refers to an experimental study in human subjects performed for purposes related to the development and submission of information under a federal law which regulates the manufacture, use or sale of drugs.

In other embodiments, the therapeutically effective amount of second agent is lower than the minimum marketed dose (for the patient's size) for use without coadministration of a second agent and/or the therapeutically effective amount of compound of Formula I is lower than the minimum marketed dose (for the patient's size) for use without coadministration of a second agent. For example, the therapeutically effective amount of one or both of second agent and compound of Formula I may be less than 95%, less than 90%, less than 75% or less than 50% of the minimum marketed dose. In certain such embodiments, the patient is a non-human animal, such as a companion animal (e.g., a dog or cat).

Within separate aspects, the present invention provides a variety of non-pharmaceutical in vitro and in vivo uses for the compounds provided herein. For example, such compounds may be labeled and used as probes for the detection and localization of CB1 (in samples such as cell preparations or tissue sections, preparations or fractions thereof). In addition, compounds provided herein that comprise a suitable reactive group (such as an aryl carbonyl, nitro or azide group) may be used in photoaffinity labeling studies of receptor binding sites. In addition, compounds provided herein may be used as positive controls in assays for receptor activity, as standards for determining the ability of a candidate agent to bind to CB1, or as radiotracers for positron emission tomography (PET) imaging or for single photon emission computerized tomography (SPECT). Such methods can be used to characterize CB1 receptors in living subjects. For example, a compound may be labeled using any of a variety of well known techniques (e.g., radiolabeled with a radionuclide such as tritium, as described herein), and incubated with a sample for a suitable incubation time (e.g., determined by first assaying a time course of binding). Following incubation, unbound compound is removed (e.g., by washing), and bound compound detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of CB1 in the sample. Detection assays, including receptor autoradiography (receptor mapping) of CB1 in cultured cells or tissue samples may be performed as described by Kuhar in sections 8.1.1 to 8.1.9 of Current Protocols in Pharmacology (1998) John Wiley & Sons, New York.

Compounds provided herein may further be used within assays for the identification of other non-competitive antagonists of CB1. In general, such assays are standard competition binding assays, in which bound, labeled compound is displaced by a test compound. Briefly, such assays are performed by: (a) contacting CB1 with a radiolabeled compound of Formula I and a test compound, under conditions that permit binding of compound to CB1 (b) removing unbound labeled compound and unbound test compound; (c) detecting a signal that corresponds to the amount of bound, labeled compound; and (d) comparing the signal to a reference signal that corresponds to the amount of bound labeled compound in a similar assay performed in the absence of test compound. In practice, the reference signal and the signal described in step (c) are generally obtained simultaneously, using assays that are identical except for the presence of test compound; in addition, multiple concentrations of test compound are generally assayed. Non-competitive antagonist activity can be confirmed for test compounds that decrease the amount of bound, labeled compound in such assays using the procedures described herein.

The following Examples are offered by way of illustration and not by way of limitation. Unless otherwise specified all reagents and solvent are of standard commercial grade and are used without further purification. Using routine modifications, the starting materials may be varied and additional steps employed to produce other compounds provided herein.

### EXAMPLES

Mass spectroscopy data in the following Examples is Electrospray MS, obtained in positive ion mode using a Micromass Time-of-Flight LCT (Micromass, Beverly Mass.), equipped with a Waters 600 pump (Waters Corp.; Milford, Mass.), Waters 996 photodiode array detector, and a Gilson 215 autosampler (Gilson, Inc.; Middleton, Wis. MassLynx (Advanced Chemistry Development, Inc; Toronto, Canada) version 4.0 software with OpenLynx Global Server™, OpenLynx™ and AutoLynx™ processing is used for data collection and analysis. MS conditions are as follows: capillary voltage=3.5 kV; cone voltage=30 V, desolvation and source temperature=350° C. and 120° C., respectively; mass range=181-750 with a scan time of 0.22 seconds and an interscan delay of 0.05 minutes.

Sample volume of 1 microliter is injected onto a 50×4.6 mm Chromolith SpeedROD RP-18e column (Merck KGaA, Darmstadt, Germany), and eluted using a 2-phase linear gradient at a flow rate of 6 ml/min. Sample is detected using total absorbance count over the 220-340 nm UV range. The elution conditions are: Mobile Phase A—95% water, 5% methanol with 0.05% TFA; Mobile Phase B—5% water, 95% methanol with 0.025% TFA. The following gradient is used: 0-0.5 minutes 10-100% B, hold at 100% B to 1.2 minutes, return to 10% B at 1.21 minutes. Inject to inject cycle is 2.15 minutes.

### Example 1

### Preparation of Representative CB1 Antagonists

This Example illustrates the preparation of representative arylalkyl ureas.

### A. N-[3-(DIFLUOROMETHOXY)PHENYL]-N′-[2-(3-FLUOROPHENYL)ETHYL]UREA

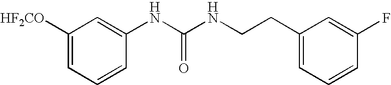

A solution of 3-fluorophenethylamine (25 mg) and 3-difluoromethoxyphenyl isocyanate (1 eq.) in toluene (1 mL) is heated at 70° C. for 1 hour. The resulting white solid is collected by suction filtration and dried with suction for 30 minutes to obtain N-[3-(difluoromethoxy)phenyl]-N′-[2-(3-fluorophenyl)ethyl]urea as a white solid.

### B. N-[3-(DIFLUOROMETHOXY)PHENYL]-N′-[2-(3,5-DIFLUOROPHENYL)ETHYL]UREA

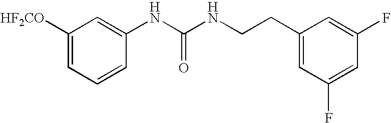

3,5-Difluorophenethylamine (100 mg, 0.64 mmol) is added to a solution of 3-difluoromethoxyphenyl isocyanate (100 mg, 0.54 mmol) in DCM (3 mL) and the mixture is stirred for 3 hours at room temperature. Hydrazine (30 μL) is added to the mixture and stirring is continued for 30 minutes at ambient temperature. The resulting mixture is concentrated under reduced pressure and purified by preparatory TLC (hexane-EtOAc, 1:1) to afford a colorless oil. The oil is dissolved in DCM (1 mL) and to this solution hexane (5 mL) is slowly added. The precipitate is filtered and dried under vacuum to afford the title product as a white solid. ¹H NMR: 7.22 (m, 1H), 7.18 (m, 1H), 7.02 (m, 1H), 6.81 (m, 1H), 6.70 (m, 3H), 6.51 (br s, 1H), 4.81 (br s, 1H), 3.48 (m, 2H), 2.81 (t, J=8 Hz, 2H).

### C. N-[3-(DIFLUOROMETHOXY)PHENYL]-N′-(2-PHENYLCYCLOPROPYL)UREA

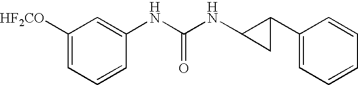

A solution of 2-phenylcyclopropylamine (25 mg) and 3-difluoromethoxyphenyl isocyanate (1 eq.) in hexane (1 mL) is shaken for 20 minutes at ambient temperature. The resulting white solid is collected by suction filtration and dried with suction to obtain N-[3-(difluoromethoxy)phenyl]-N′-(2-phenylcyclopropyl)urea as a white solid. ¹H NMR: CDCl₃ (300 MHz) δ 7.19-7.35 (m, 5H), 7.05-7.11 (m, 4H), 6.75-6.79 (m, 1H), 6.47 (t, J=75 Hz, 1H), 5.41 (Br, 1H), 2.71 (m, 1H), 2.15 (m, 1H), 1.33 (m, 2H).

### Example 2

### High Speed Synthesis of Representative CB1 Antagonists

This Example illustrates the high speed synthesis of representative arylalkyl ureas provided herein.
A. Protocol 1

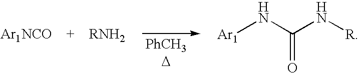

Aryl isocyanate (0.15 mL of 0.2 M in toluene) is added to a reaction vial followed by alkyl amine (0.1 mL of 0.2M in toluene). The reaction vessel is sealed and heated at 70° C. with agitation for 16 hours. A solution of N-(3-aminopropyl)morpholine (0.5 mL of 0.2 M in ethyl acetate) is added and the reaction is heated at 70° C. for 1 hour. The reaction is cooled, diluted with ethyl acetate (0.3 mL) and eluted through a silica gel SPE cartridge with ethyl acetate (3.0 mL). The eluent is evaporated, weighed and diluted to a concentration of 10 mM in DMSO. Purity is assessed using LC/MS.
B. Protocol 2—Curtius Rearrangement

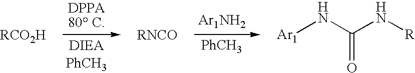

To a solution of alkyl carboxylic acid (0.15 mL of 0.2 M in toluene/5% vv DIEA) is added diphenylphosphoryl azide (0.12 mL of 2M in toluene). The reaction vessel is sealed and heated at 80° C. for 4 hours with agitation. The reaction mixture is cooled, aryl amine (0.1 mL of 0.2 M in toluene) is added and the reaction mixture is agitated at room temperature for 1 hour. The reaction mixture is partitioned between ethyl acetate (0.5 mL) and 1 N NaOH. The upper phase is removed and purified on a SCX cartridge eluting with 25% MeOH/EtOAc (3 mL). The eluent is evaporated, weighed and diluted to a concentration of 10 mM in DMSO. Purity is assessed using LC/MS.

### Example 3

### Additional Representative CB1 Antagonists

Using routine modifications, the starting materials may be varied and additional steps employed to produce other compounds provided herein. Compounds listed in Table I are prepared using such methods. For all compounds in Table 1, the IC₅₀ at CB1, determined as described in Example 8 herein, is 2 micromolar or less. “R.T.” is the retention time in minutes and mass spec data (MS) was generated as described above and is presented as M+1.

TABLE 1

| | Compound | Name | R.T. | MS |
|---|---|---|---|---|
| 1 | 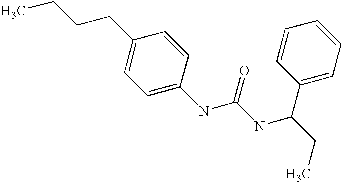 | N-(4-butylphenyl)-N′-(1- phenylpropyl)urea | 1.27 | 311.3 |
| 2 | 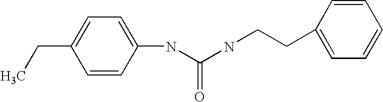 | N-(4-ethylphenyl)-N′-(2- phenylethyl)urea | 1.32 | 269.2 |
| 3 | 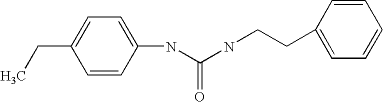 | N-(4-ethylphenyl)-N′-(2- phenylethyl)urea | 1.19 | 269.2 |
| 4 | 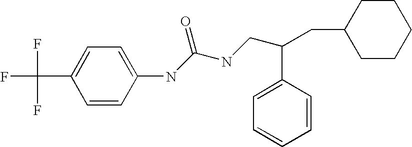 | N-(3-cyclohexyl-2-phenylpropyl)-N′- [4-(trifluoromethyl)phenyl]urea | 1.36 | 405.2 |
| 5 | 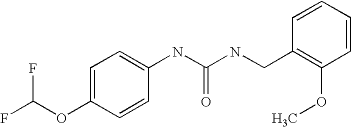 | N-[4-(difluoromethoxy)phenyl]-N′-(2- methoxybenzyl)urea | 1.26 | 323.1 |
| 6 | 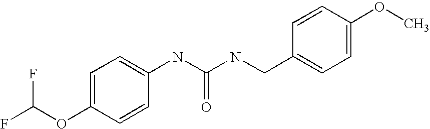 | N-[4-(difluoromethoxy)phenyl]-N′-(4- methoxybenzyl)urea | | |
| 7 | 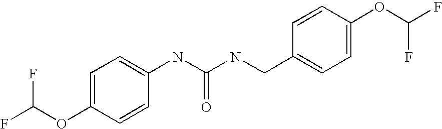 | N-[4-(difluoromethoxy)benzyl]-N′-[4- (difluoromethoxy)phenyl]urea | 1.27 | 359.1 |
| 8 | 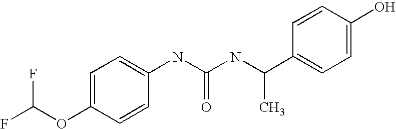 | N-[4-(difluoromethoxy)phenyl]-N′-[1- (4-hydroxyphenyl)ethyl]urea | 1.20 | 323.1 |
| 9 | 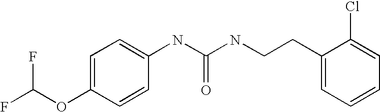 | N-[2-(2-chlorophenyl)ethyl]-N′-[4- (difluoromethoxy)phenyl]urea | 1.31 | 341.1 |
| 10 | 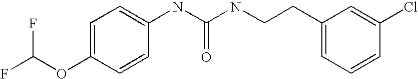 | N-[2-(3-chlorophenyl)ethyl]-N′-[4- (difluoromethoxy)phenyl]urea | 1.32 | 341.1 |
| 11 | 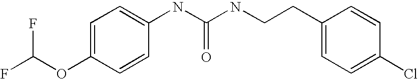 | N-[2-(4-chlorophenyl)ethyl]-N′-[4- (difluoromethoxy)phenyl]urea | 1.32 | 341.1 |
| 12 | 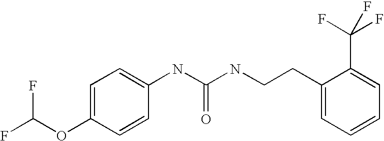 | N-[4-(difluoromethoxy)phenyl]-N′-{2- [2-(trifluoromethyl)phenyl]ethyl}urea | | |
| 13 | 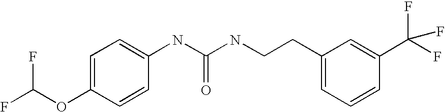 | N-[4-(difluoromethoxy)phenyl]-N′-{2- [3-(trifluoromethyl)phenyl]ethyl}urea | | |
| 14 | 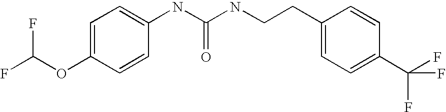 | N-[4-(difluoromethoxy)phenyl]-N′-{2- [4-(trifluoromethyl)phenyl]ethyl}urea | 1.33 | 375.1 |
| 15 | 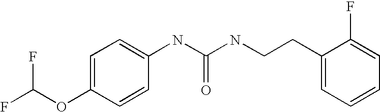 | N-[4-(difluoromethoxy)phenyl]-N′-[2- (2-fluorophenyl)ethyl]urea | 1.28 | 325.1 |
| 16 | 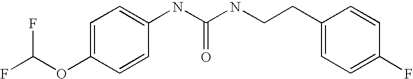 | N-[4-(difluoromethoxy)phenyl]-N′-[2- (4-fluorophenyl)ethyl]urea | 1.28 | 325.1 |
| 17 | 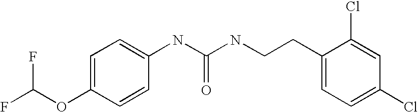 | N-[2-(2,4-dichlorophenyl)ethyl]-N′-[4- (difluoromethoxy)phenyl]urea | | |
| 18 | 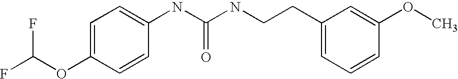 | N-[4-(difluoromethoxy)phenyl]-N′-[2- (3-methoxyphenyl)ethyl]urea | | |
| 19 | 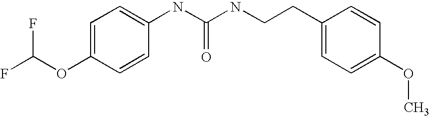 | N-[4-(difluoromethoxy)phenyl]-N′-[2- (4-methoxyphenyl)ethyl]urea | 1.28 | 337.1 |
| 20 | 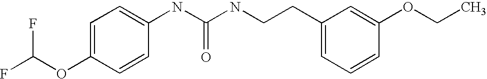 | N-[4-(difluoromethoxy)phenyl]-N′-[2- (3-ethoxyphenyl)ethyl]urea | | |
| 21 | 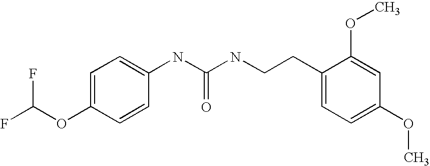 | N-[4-(difluoromethoxy)phenyl]-N′-[2- (2,4-dimethoxyphenyl)ethyl]urea | 1.29 | 367.1 |
| 22 | 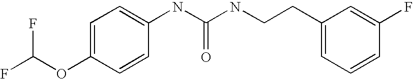 | N-[4-(difluoromethoxy)phenyl]-N′-[2- (3-fluorophenyl)ethyl]urea | | |
| 23 | 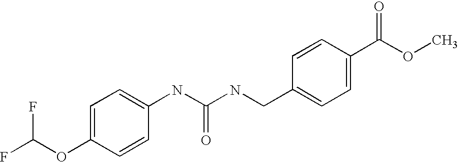 | methyl 4-{[({[4- (difluoromethoxy)phenyl]amino}car- bonyl)amino]methyl}benzoate | 1.25 | 351.1 |
| 24 | 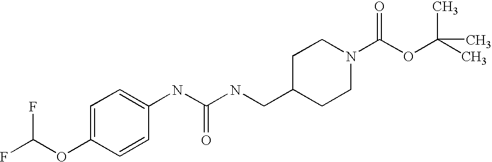 | tert-butyl 4-{[({[4- (difluoromethoxy)phenyl]amino}car- bonyl)amino]methyl}piperidine-1- carboxylate | 1.31 | 344.1 |
| 25 | 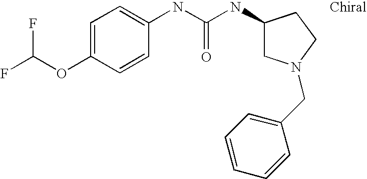 | N-[(3S)-1-benzylpyrrolidin-3-yl]-N′- [4-(difluoromethoxy)phenyl]urea | 1.16 | 362.2 |
| 26 | 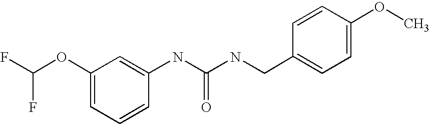 | N-[3-(difluoromethoxy)phenyl]-N′-(4- methoxybenzyl)urea | 1.25 | 323.1 |
| 27 | 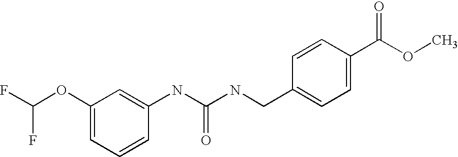 | methyl 4-{[({[3- (difluoromethoxy)phenyl]amino}car- bonyl)amino]methyl}benzoate | 1.25 | 351.2 |
| 28 | 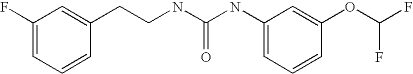 | N-[3-(difluoromethoxy)phenyl]-N′-[2- (3-fluorophenyl)ethyl]urea | 1.29 | 325.1 |
| 29 | 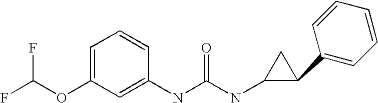 | N-[3-(difluoromethoxy)phenyl]-N′-(2- phenylcyclopropyl)urea | 1.29 | 319.1 |
| 30 | 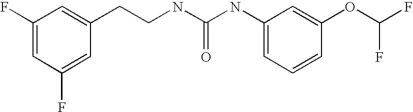 | N-[3-(difluoromethoxy)phenyl]-N′-[2- (3,5-difluorophenyl)ethyl]urea | | |
| 31 | 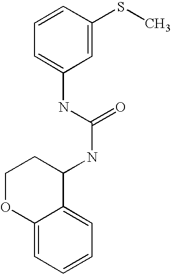 | N-(3,4-dihydro-2H-chromen-4-yl)-N′- [3-(methylthio)phenyl]urea | | |
| 32 | 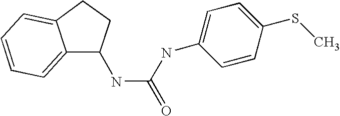 | N-(2,3-dihydro-1H-inden-1-yl)-N′-[4- (methylthio)phenyl]urea | | |
| 33 | 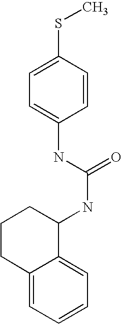 | N-[4-(methylthio)phenyl]-N′-(1,2,3,4- tetrahydronaphthalen-1-yl)urea | | |
| 34 | 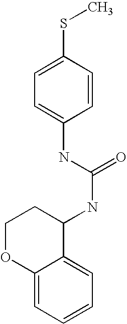 | N-(3,4-dihydro-2H-chromen-4-yl)-N′- [4-(methylthio)phenyl]urea | | |
| 35 | 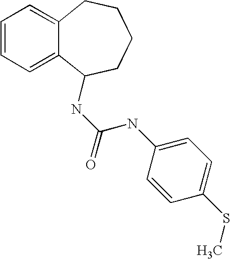 | N-[4-(methylthio)phenyl]-N′-(6,7,8,9- tetrahydro-5H-benzo[7]annulen-5- yl)urea | | |
| 36 | 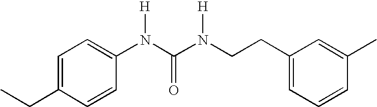 | N-(4-ethylphenyl)-N′-[2-(3- methylphenyl)ethyl]urea | | |
| 37 | 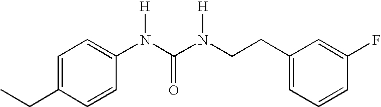 | N-(4-ethylphenyl)-N′-[2-(3- fluorophenyl)ethyl]urea | | |
| 38 | 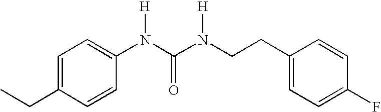 | N-(4-ethylphenyl)-N′-[2-(4- fluorophenyl)ethyl]urea | | |
| 39 | 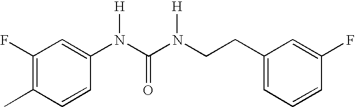 | N-(3-fluoro-4-methylphenyl)-N′-[2-(3- fluorophenyl)ethyl]urea | | |
| 40 | 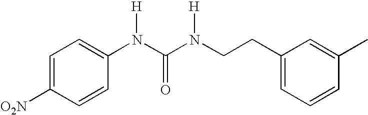 | N-[2-(3-methylphenyl)ethyl]-N′-(4- nitrophenyl)urea | 2.12 | 300.2 |
| 41 | 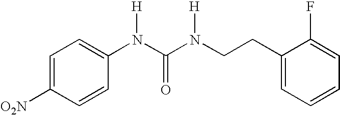 | N-[2-(2-fluorophenyl)ethyl]-N′-(4- nitrophenyl)urea | 2.05 | 304.2 |
| 42 | 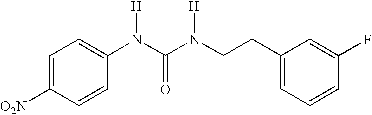 | N-[2-(3-fluorophenyl)ethyl]-N′-(4- nitrophenyl)urea | 2.07 | 304.2 |
| 43 | 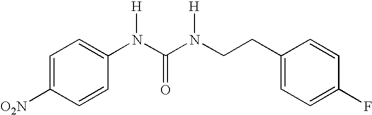 | N-[2-(4-fluorophenyl)ethyl]-N′-(4- nitrophenyl)urea | 2.07 | 304.2 |
| 44 | 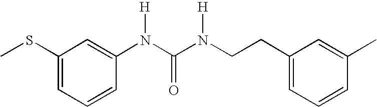 | N-[2-(3-methylphenyl)ethyl]-N′-[3- (methylthio)phenyl]urea | 2.14 | 301.2 |
| 45 | 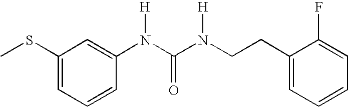 | N-[2-(2-fluorophenyl)ethyl]-N′-[3- (methylthio)phenyl]urea | 2.07 | 305.2 |
| 46 | 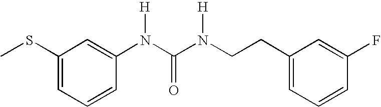 | N-[2-(3-fluorophenyl)ethyl]-N′-[3- (methylthio)phenyl]urea | 2.08 | 305.2 |
| 47 | 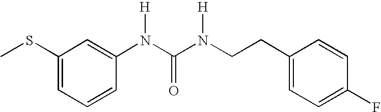 | N-[2-(4-fluorophenyl)ethyl]-N′-[3- (methylthio)phenyl]urea | 2.08 | 305.2 |
| 48 | 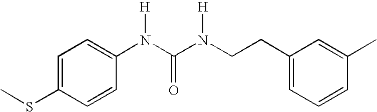 | N-[2-(3-methylphenyl)ethyl]-N′-[4- (methylthio)phenyl]urea | 2.12 | 301.2 |
| 49 | 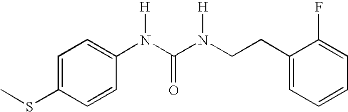 | N-[2-(2-fluorophenyl)ethyl]-N′-[4- (methylthio)phenyl]urea | 2.06 | 305.2 |
| 50 | 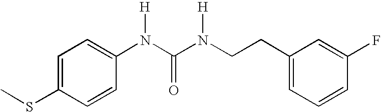 | N-[2-(3-fluorophenyl)ethyl]-N′-[4- (methylthio)phenyl]urea | 2.07 | 305.2 |
| 51 | 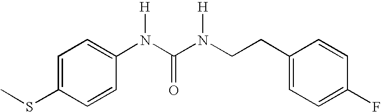 | N-[2-(4-fluorophenyl)ethyl]-N′-[4- (methylthio)phenyl]urea | 2.07 | 305.2 |
| 52 | 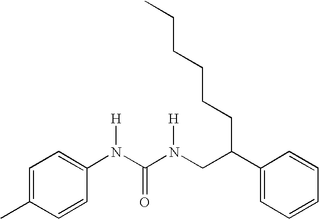 | N-(4-methylphenyl)-N-(2- phenyloctyl)urea | | |
| 53 | 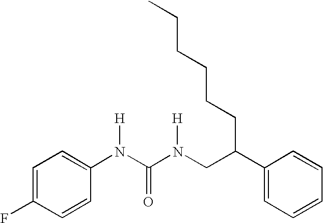 | N-(4-fluorophenyl)-N′-(2- phenyloctyl)urea | | |
| 54 | 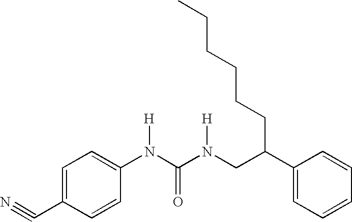 | N-(4-cyanophenyl)-N′-(2- phenyloctyl)urea | | |
| 55 | 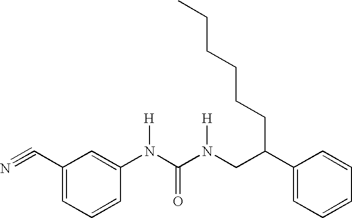 | N-(3-cyanophenyl)-N′-(2- phenyloctyl)urea | | |
| 56 | 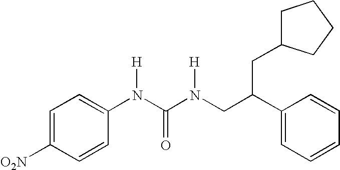 | N-(3-cyclopentyl-2-phenylpropyl)-N- (4-nitrophenyl)urea | | |
| 57 | 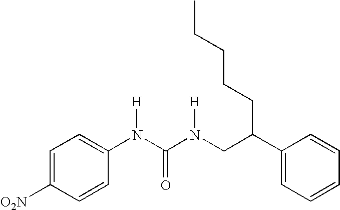 | N-(4-nitrophenyl)-N′-(2- phenylheptyl)urea | | |
| 58 | 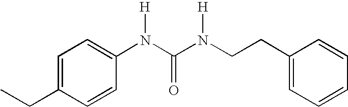 | 1-(4-ethylphenyl)-3-(2- phenylethyl)urea | 1.23 | 269.2 |
| 59 | 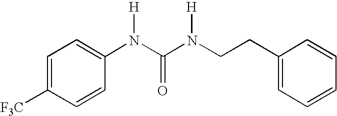 | 1-(2-phenylethyl)-3-[4- (trifluoromethyl)phenyl]urea | 1.26 | 309.1 |
| 60 | 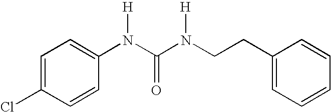 | 1-(4-chlorophenyl)-3-(2- phenylethyl)urea | 1.3 | 276.1 |
| 61 | 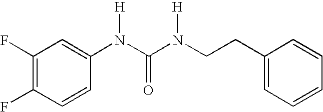 | 1-(3,4-difluorophenyl)-3-(2- phenylethyl)urea | 1.29 | 277.2 |
| 62 | 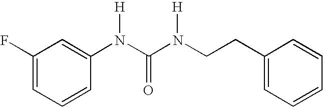 | 1-(3-fluorophenyl)-3-(2- phenylethyl)urea | 1.27 | 259.1 |
| 63 | 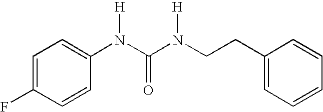 | 1-(4-fluorophenyl)-3-(2- phenylethyl)urea | 1.26 | 259.1 |
| 64 | 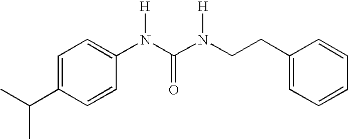 | 1-(4-isopropylphenyl)-3-(2- phenylethyl)urea | 1.34 | 283.2 |
| 65 | 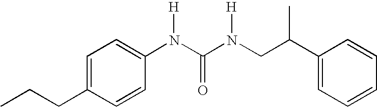 | 1-(2-phenylpropyl)-3-(4- propylphenyl)urea | 1.37 | 297.2 |
| 66 | 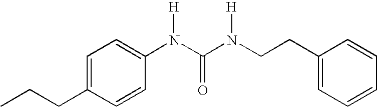 | 1-(2-phenylethyl)-3-(4- propylphenyl)urea | 1.35 | 283.2 |
| 67 | 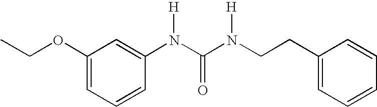 | 1-(3-ethoxyphenyl)-3-(2- phenylethyl)urea | 1.28 | 285.2 |
| 68 | 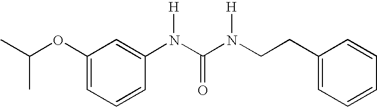 | 1-(3-isopropoxyphenyl)-3-(2- phenylethyl)urea | 1.31 | 299.2 |
| 69 | 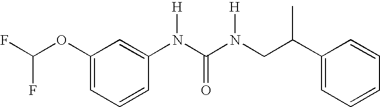 | 1-[3-(difluoromethoxy)phenyl]-3-(2- phenylpropyl)urea | 1.3 | 321.2 |
| 70 | 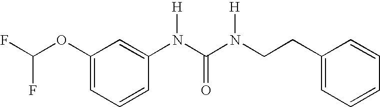 | 1-[3-(difluoromethoxy)phenyl]-3-(2- phenylethyl)urea | 1.28 | 307.2 |
| 71 | 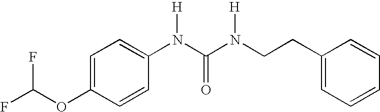 | 1-{4-(difluoromethoxy)phenyl]-3-(2- phenylethyl)urea | 1.27 | 307.2 |
| 72 | 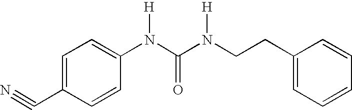 | 1-[4-(cyanomethyl)phenyl]-3-(2- phenylethyl)urea | 1.22 | 280.1 |
| 73 | 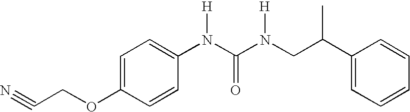 | 1-[4-(cyanomethoxy)phenyl]-3-(2- phenylpropyl)urea | 1.24 | 310.1 |
| 74 | 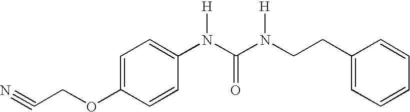 | 1-[4-(cyanomethoxy)phenyl]-3-(2- phenylethyl)urea | 1.22 | 296.1 |
| 75 | 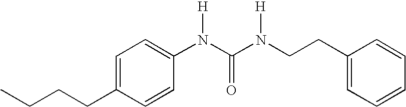 | 1-(4-butylphenyl)-3-(2- phenylethyl)urea | 1.31 | 297.2 |
| 76 | 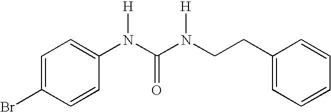 | 1-(4-bromophenyl)-3-(2- phenylethyl)urea | 1.25 | 319.1 |
| 77 | 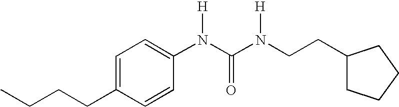 | 1-(4-butylphenyl)-3-(2- cyclopentylethyl)urea | 1.34 | 289.2 |
| 78 | 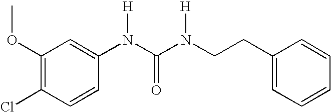 | 1-(4-chloro-3-methoxyphenyl)-3-(2- phenylethyl)urea | 1.22 | 305.1 |
| 79 | 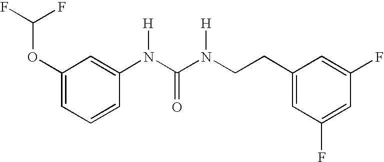 | N-[3-(difluoromethoxy)phenyl]-N′-[2- (3,5-difluorophenyl)ethyl]urea | 1.22 | 343.2 |
| 80 | 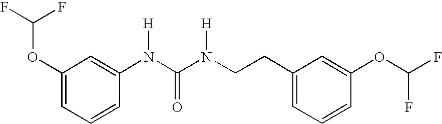 | N-[3-(difluoromethoxy)phenyl]-N′-{2- [3-(difluoromethoxy)phenyl]ethyl}urea | 1.29 | 373.1 |
| 81 | 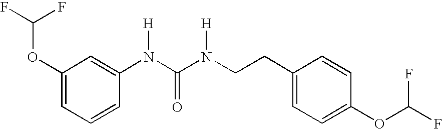 | N-[3-(difluoromethoxy)phenyl]-N′-{2- [4-(difluoromethoxy)phenyl]ethyl}urea | 1.28 | 373.1 |
| 82 | 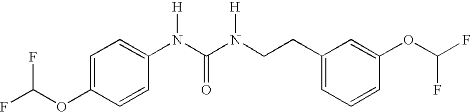 | N-[4-(difluoromethoxy)phenyl]-N′-{2- [3-(difluoromethoxy)phenyl]ethyl}urea | 1.21 | 373.2 |

### Example 4

### Baculoviral Preparations for CB1 Expression

This Example illustrates the preparation of recombinant baculovirus for use in generating CB1-expressing insect cells.

The human CB1 sequence has GenBank Accession Number HSU73304, and was reported by Hoehe et al. (1991) New Biol. 3(9):880-85. Human CB1 (hCB1) cDNA is amplified from a human brain cDNA library (Gibco BRL, Gaithersburg, Md.) using PCR, in which the 5′ primer includes the optimal Kozak sequence CCACC. The resulting PCR product is cloned into pcDNA3.1/V5-His-TOPO (Invitrogen Corp, Carlsbad, Calif.) using the multiple cloning site, and then subcloned into pBACPAK₈ (BD Biosciences, Palo Alto, Calif.) at the Bam/Xho site to yield a hCB1 baculoviral expression vector.

The hCB1 baculoviral expression vector is co-transfected along with BACULOGOLD DNA (BD PharMingen, San Diego, Calif.) into Sf9 cells. The Sf9 cell culture supernatant is harvested three days post-transfection. The recombinant virus-containing supernatant is serially diluted in Hink's TNM-FH insect medium (JRH Biosciences, Kansas City, Mo.) supplemented with Grace's salts and with 4.1 mM L-Gln, 3.3 g/L LAH, 3.3 g/L ultrafiltered yeastolate and 10% heat-inactivated fetal bovine serum (hereinafter “insect medium”) and plaque assayed for recombinant plaques. After four days, recombinant plaques are selected and harvested into 1 ml of insect medium for amplification. Each 1 ml volume of recombinant baculovirus (at passage 0) is used to infect a separate T25 flask containing 2×10⁶ Sf9 cells in 5 ml of insect medium. After five days of incubation at 27° C., supernatant medium is harvested from each of the T25 infections for use as passage 1 inoculum.

Two of seven recombinant baculoviral clones are then chosen for a second round of amplification, using 1 ml of passage 1 stock to infect 1×10⁸ cells in 100 ml of insect medium divided into 2 T175 flasks. Forty-eight hours post infection, passage 2 medium from each 100 ml preparation is harvested and plaque assayed for titer. The cell pellets from the second round of amplification are assayed by affinity binding as described below to verify recombinant receptor expression. A third round of amplification is then initiated using a multiplicity of infection of 0.1 to infect a liter of Sf9 cells. Seventy-two hours post-infection the supernatant medium is harvested to yield passage 3 baculoviral stock.

The remaining cell pellet is assayed for affinity binding. Radioligand is 25 pM-5.0 nM [³H]CP55,940 for saturation binding and 0.5 nM for competition binding (New England Nuclear Corp., Boston, Mass.); the hCB1-expressing baculoviral cells are used; the assay buffer contains 50 mM Tris pH 7.4, 120 mM NaCl, 5 mM MgCl₂, 0.5% BSA and 0.2 mg/ml bacitracin; filtration is carried out using GF/C WHATMAN filters (presoaked in 0.3% non-fat dry milk (H₂O) for 2 hours prior to use); and the filters are washed twice with 5 mL cold 50 mM Tris pH.7.4.

Titer of the passage 3 baculoviral stock is determined by plaque assay and a multiplicity of infection, incubation time course, binding assay experiment is carried out to determine conditions for optimal receptor expression.

### Example 5

### Baculoviral Infections

Log-phase Sf9 cells (Invitrogen Corp.; Carlsbad, Calif.), are infected with one or more stocks of recombinant baculovirus followed by culturing in insect medium at 27° C. Infections are carried out either only with virus directing the expression of hCB1 or with this virus in combination with three G-protein subunit-expression virus stocks: 1) rat Gaα₁₂ G-protein-encoding virus stock, 2) bovine β1 G-protein-encoding virus stock, and 3) human γ2 G-protein-encoding virus stock, all of which are obtained from Biosignal Inc., Montreal, Canada.

Typical hCB1 infections are conducted using Sf9 cells that are cultured in insect medium supplemented with 10% heat-inactivated fetal bovine serum (FBS) as discussed above. Higher receptor and G-protein (Gα, Gβ, Gγ) expression can be obtained if the Sf9 cells are cultured in insect medium with 5% FBS and 5% Gibco serum-free medium (Invitrogen Corp.; Carlsbad, Calif.). Maximal CB1 receptor expression and functional activity is achieved if the Sf9 cells are cultured in insect medium without FBS and with 10% Gibco serum-free medium. The infections are carried out at a multiplicity of infection of 0.1:1.0:0.5:0.5. At 72 hours post-infection, a sample of cell suspension is analyzed for viability by trypan blue dye exclusion, and the remaining Sf9 cells are harvested via centrifugation (3000 rpm/10 minutes/4° C.).

### Example 6

### Purified Recombinant Insect Cell Membranes

Sf9 cell pellets are resuspended in homogenization buffer (10 mM HEPES, 250 mM sucrose, 0.5 μg/ml leupeptin, 2 μg/ml Aprotinin, 200 μM PMSF, and 2.5 mM EDTA, pH 7.4) and homogenized using a POLYTRON homogenizer (setting 5 for 30 seconds). The homogenate is centrifuged (536×g/10 minutes/4° C.) to pellet the nuclei. The supernatant containing isolated membranes is decanted to a clean centrifuge tube, centrifuged (48,000×g/30 minutes, 4° C.) and the resulting pellet resuspended in 30 ml homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is measured using a Bradford protein assay (Bio-Rad Laboratories, Hercules, Calif.). By this measure, a 1-liter culture of cells typically yields 100-150 mg of total membrane protein.

### Example 7

### Radioligand Binding Assays

P2 membranes are resuspended by Dounce homogenization (tight pestle) in binding buffer (50 mM Tris pH. 7.4, 120 mM NaCl, 5 mM MgCl₂, 0.5% BSA and 0.2 mg/ml bacitracin).

For saturation binding analysis, membranes (10 μg) are added to polypropylene tubes containing 25 pM-0.5 nM [³H]CP55,940 (New England Nuclear Corp., Boston, Mass.). Nonspecific binding is determined in the presence of 10 μM CP55,940 (Tocris Cookson Inc., Ellisville, Mo.) and accounted for less than 10% of total binding. For evaluation of guanine nucleotide effects on receptor affinity, GTPγS is added to duplicate tubes at the final concentration of 50 μM.

For competition analysis, membranes (10 μg) are added to polypropylene tubes containing 0.5 nM [³H]CP55,940. Non-radiolabeled displacers are added to separate assays at concentrations ranging from 10⁻¹⁰ M to 10⁻⁵ M to yield a final volume of 0.250 mL. Nonspecific binding is determined in the presence of 10 μM CP55,940 and accounted for less than 10% of total binding. Following a one-hour incubation at room temperature, the reaction is terminated by rapid vacuum filtration. Samples are filtered over presoaked (0.3% non-fat dry milk for 2 hours prior to use) GF/C WHATMAN filters and rinsed 2 times with 5 mL cold 50 mM Tris pH 7.4. Remaining bound radioactivity is quantified by gamma counting. K_{I} and Hill coefficient (“nH”) are determined by fitting the Hill equation to the measured values with the aid of SIGMAPLOT software (SPSS Inc., Chicago, Ill.).

### Example 8

### Agonist-Induced GTP Binding

This Example illustrates the use of agonist-stimulated GTP-gamma³⁵S binding (“GTP binding”) activity to identify CB1 agonists and antagonists, and to differentiate neutral antagonists from those that possess inverse agonist activity. This assay can also be used to detect partial agonism mediated by antagonist compounds. A compound being analyzed in this assay is referred to herein as a “test compound.” Agonist-stimulated GTP binding activity is measured as follows: Four independent baculoviral stocks (one directing the expression of hCB1 and three directing the expression of each of the three subunits of a heterotrimeric G-protein) are used to infect a culture of Sf9 cells as described in Example 5.

Agonist-stimulated GTP binding on purified membranes (prepared as described in Example 6) is initially assessed using the CB1 agonist CP55,940 to ascertain that the receptor/G-protein-alpha-beta-gamma combination(s) yield a functional response as measured by GTP binding.

P2 membranes are resuspended by Dounce homogenization (tight pestle) in GTP binding assay buffer (50 mM Tris pH 7.4, 120 mM NaCl, 5 mM MgCl₂, 2 mM EGTA, 0.1% BSA, 0.1 mM bacitracin, 100KIU/mL aprotinin, 5 μM GDP) and added to reaction tubes at a concentration of 10 μg protein/reaction tube. After adding increasing doses of the agonist CP55,940 at concentrations ranging from 10⁻¹² M to 10⁻⁶M, reactions are initiated by the addition of 100 μM GTPgamma³⁵S. In competition experiments, non-radiolabeled test compounds are added to separate assays at concentrations ranging from 10⁻¹⁰ M to 10⁻⁵ M along with 1 nM CP55,940 to yield a final volume of 0.25 mL.

Following a 60-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTPgamma³⁵S is determined by measuring the bound radioactivity, preferably by liquid scintillation spectrometry of the washed filters. Non-specific binding is determined using 10 mM GTPgamma³⁵S and typically represents less than 5% of total binding. Data is expressed as percent above basal (baseline). The results of these GTP binding experiments are analyzed using SIGMAPLOT software and IC₅₀ determined. The IC₅₀ may then be used to generate K; as described by Cheng and Prusoff (1973) Biochem Pharmacol. 22(23):3099-108.

Neutral antagonists are those test compounds that reduce the CP55,940-stimulated GTP binding activity towards, but not below, baseline (the level of GTP bound by membranes in this assay in the absence of added CP55,940 or other agonist and in the further absence of any test compound).

In contrast, in the absence of added CP55,940, CB1 inverse agonists reduce the GTP binding activity of the receptor-containing membranes below baseline. If a test compound that displays antagonist activity does not reduce the GTP binding activity below baseline in the absence of the CB1 agonist, it is characterized as a neutral antagonist.

An antagonist test compound that elevates GTP binding activity above baseline in the absence of added CP55,940 in this GTP binding assay is characterized as having partial agonist activity. Preferred CB1 antagonists do not elevate GTP binding activity under such conditions more than 10%, more preferably less than 5% and most preferably less than 2% of the maximal response elicited by the agonist, CP55,940.

The GTP binding assay can also be used to determine antagonist selectivity towards the CB1 receptor over the CB2 receptor. Agonist-stimulated GTP binding activity at the CB2 receptor is measured as described above for CB1, except that hCB2 is used in place of hCB1. Results in such assays are then compared to the results in assays employing hCB1 to assess selectivity.

### Example 9

### Surmountability Assays

Certain CB1 receptor antagonists are insurmountable with regard to the agonist induced GTPγ³⁵S binding effect. To assess surmountability, P2 membranes are resuspended by Dounce homogenization (tight pestle) in GTP binding assay buffer (50 mM Tris pH 7.4, 120 mM NaCl, 5 mM MgCl₂, 2 mM EGTA, 10 μg/ml saponin, 0.1% BSA, 0.1 mM bacitracin, 100KIU/mL aprotinin, 5 μM GDP) and added to reaction tubes at a concentration of 10 μg protein/reaction tube. Agonist dose-response curves (typically CP55,940) at concentrations ranging from 10⁻¹² M to 10⁻⁵ M, are run either in the absence or in the presence of a test compound at one of several doses up to 100× the IC₅₀ of the test compound as measured in the competition GTPγ³⁵S binding. The reactions are initiated by the addition of 100 μM GTPγ³⁵S to yield a final volume of 0.25 mL. Following a 90-minute incubation at room temperature, the reactions are terminated by vacuum filtration over GF/C filters (pre-soaked in wash buffer, 0.1% BSA) followed by washing with ice-cold wash buffer (50 mM Tris pH 7.0, 120 mM NaCl). The amount of receptor-bound (and thereby membrane-bound) GTPγ³⁵S is determined by measuring the bound radioactivity, preferably by liquid scintillation spectrometry of the washed filters. Non-specific binding is determined using 10 μM GTPγS and typically represents less than 5 percent of total binding. Data is expressed as percent above basal (baseline). The results of these GTP binding experiments may be conveniently analyzed using SIGMAPLOT software. A surmountable test compound is one which shifts the EC₅₀ of the agonist to the right (weaker) without affecting the maximum functional response of the agonist. Insurmountable antagonist test compounds have no significant effect on the hCB1 agonist EC₅₀ at concentrations roughly 100 times the IC₅₀, but significantly reduce or eliminate the agonist stimulated GTPγ³⁵S binding response of the receptor.

### Example 10

### MDCK Cytotoxicity Assay

This Example illustrates the evaluation of compound toxicity using a Madin Darby canine kidney (MDCK) cell cytotoxicity assay.

1 μL of test compound is added to each well of a clear bottom 96-well plate (Packard, Meriden, Conn.) to give final concentration of compound in the assay of 10 μM, 100 μM or 200 μM. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/mL with warm (37° C.) medium (VITACELL Minimum Essential Medium Eagle, ATCC catalog #30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution (from the Packard (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM, and 12.5 nM. PACKARD substrate solution (50 mL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A method for treating a condition in a patient, wherein the condition is an appetite disorder, obesity, an addictive disorder, asthma, liver cirrhosis, sepsis, irritable bowel disease, Crohn's disease, depression, schizophrenia, a memory disorder, a cognitive disorder, a movement disorder or bone loss, the method comprising administering to the patient a therapeutically effective amount of at least one compound of the formula: 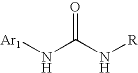
or a pharmaceutically acceptable salt thereof wherein:
R is:
(a) (5- or 6-membered heteroaryl)C₁-C₆alkyl or phenylC₁-C₆alkyl, each of which is substituted with from 0 to 4 substituents that are independently chosen from Rₓ; or
(b) C₃-C₈cycloalkyl)C₀-C₆alkyl or (3- to 8-membered heterocycloalkyl)C₀-C₆alkyl, each of which is
(i) optionally fused to a 5- to 7-membered carbocyclic or heterocyclic ring, and
(ii) substituted with from 0 to 4 substituents that are independently chosen from R^{x};
Ar₁ is phenyl, naphthyl or a 5- to 10-membered heteroaryl, each of which is substituted with from 0 to 4 substituents that are independently chosen from R_{y};
Each Rₓ is independently:
(a) hydroxy, halogen, amino, cyano, nitro, aminocarbonyl, aminosulfonyl or —COOH;
(b) a group of the formula L-M₁-Q₁, wherein:
L is absent or C₀-C₄alkylene;
M₁ is absent, O, OC(═O), OC(═O)O, N(R_{z}), C(═O)N(R_{z}), C(═NH)N(R_{z}), N(R_{z})C(═O), N(R_{z})C(═NH), N(R_{z})C(═O, OC(═O)N(R_{z}), N(R))S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen, C₁-C₈alkyl and groups that are taken together with Q₁ to for an optionally substituted 4- to 7-membered heterocycle; and
Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-membered heterocycle,
each of which is substituted with from 0 to 3 substituents independently chosen from hydroxy, halogen, amino, cyano, oxo, C₁-C₄alkyl, C₁-C₄alkoxy and C₁-C₄haloalkyl;
such that Rₓ is not unsubstituted benzyl; or
(c) taken together with an Rₓ located on an adjacent ring carbon atom to form a fused 5- to 7-membered carbocycle or heterocycle that is substituted with from 0 to 3 substituents independently chosen from hydroxy, halogen, amino, cyano, oxo, C₁-C₄alkyl, C₁-C₄alkoxy, C₁-C₄haloalkyl and C₁-C₄alkylsulfonyl; and
Each R_{y} is independently:
(a) hydroxy, halogen, amino, cyano, nitro, aminocarbonyl, aminosulfonyl or —COOS;
(b) a group of the formula L-M₂-Q₂, wherein:
L is absent or C₀-C₄allylene;
M₂ is absent, O, C(═O), OC(═O), C(═O)O, O—C(═O)O, S(O)ₘ, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen and C₁-C₈alkyl; and
Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl.

2. A method according to claim 1, wherein Ar₁ is phenyl, pyridyl or pyrimidyl, each of which is substituted with from 0 to 3 substituents independently located meta or para to the point of attachment, wherein each substituent is independently:
(a) halogen, hydroxy or cyano; or
(b) C₁-C₄alkyl, C₁-C₄alkoxy, C₁-C₄alkylthio, C₁-C₄haloalkyl, C₁-C₄haloalkoxy, C₁-C₄alkanoyl, C₁-C₄alkanoyloxy or C₁-C₄alkoxycarbonyl, each of which is substituted with from 0 to 2 substituents independently chosen from hydroxy, halogen, C₁-C₄alkyl, C₁-C₄alkoxy, C₂-C₄alkanoyl, and C₁-C₄haloalkyl.

3. A method according to claim 2, wherein Ar₁ is phenyl that is unsubstituted or substituted with 1 or 2 substituents, each of which is located meta or para to the point of attachment, and each of which is independently halogen, cyano, nitro, C₁-C₄alkyl, C₁-C₄alkoxy, C₁-C₄alkylthio, C₁-C₄haloalkyl or C₁-C₄haloalkoxy.

4. A method according to claim 1, wherein R is phenylC₁-C₄alkyl that is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

5. A method according to claim 1, wherein R is (C₃-C₈cycloalkyl)C₀-C₄alkyl or (3- to 8-membered heterocycloalkyl)C₀-C₄alkyl, each of which is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl, C₁-C₂haloalkoxy, phenylC₀-C₄alkyl and (6-membered heteroaryl)C₀-C₄alkyl.

6. A method according to claim 1, wherein R is (C₃-C₈cycloalkyl)C₀-C₄alkyl or (3- to 8-membered heterocycloalkyl)C₀-C₄alkyl, each of which is fused to a phenyl or 6-membered heteroaryl ring and each of which is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

7. A method according to claim 1, wherein the compound has the formula: 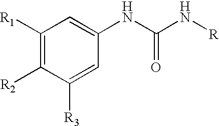
wherein R₁, R₂ and R₃ are independently chosen from hydrogen and R_{y}, such that at least one of R₁ and R₂ is not hydrogen.

8. A method according to claim 7, wherein R₁, R₂, and R₃ are independently chosen from hydrogen, halogen, cyano, C₁-C₄alkyl, C₁-C₄haloalkyl, C₁-C₄alkoxy, C₁-C₄haloalkoxy, C₁-C₄alkanoyloxy and C₁-C₄alkoxycarbonyl.

9. A method according to claim 7, wherein the compound has the formula: 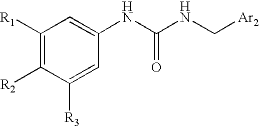
wherein Ar₂ is phenyl or a 6-membered heteroaryl, each of which is substituted with from 1 to 3 substituents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl and C₁-C₂haloalkoxy.

10. A method according to claim 7, wherein the compound has the formula: 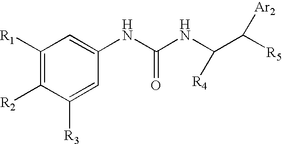
wherein:
R₄ and R₅ are independently chosen from hydrogen, C₁-C₆alkyl and (C₃-C₈cycloalkyl)C₀-C₄alkyl; or R₄ and R₅ are taken together to form a C₃-C₆cycloalkyl group; and
Ar₂ is phenyl or a 6-membered heteroaryl, each of which is substituted with from 0 to 3 substituents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl, C₁-C₂haloalkyl and C₁-C₂haloalkoxy.

11. A method according to claim 10, wherein the compound has the formula: 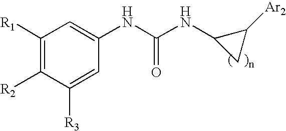
wherein n is 1, 2, 3 or 4.

12. A method according to claim 10, wherein the compound has the formula: 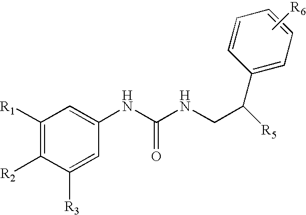
wherein:
R₅ is hydrogen, C₁-C₆alkyl or (C₃-C₈cycloalkyl)C₀-C₄alkyl; and
R₆ represents from 0 to 3 substituents independently chosen from hydroxy, halogen, C₁-C₆alkyl, C₁-C₄alkoxy, C₁-C₂haloalkyl and C₁-C₂haloalkoxy.

13. A method according to claim 7, wherein the compound has the formula: 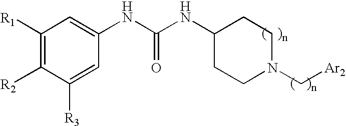
wherein:
Ar₂ is phenyl or a 6-membered heteroaryl, each of which is substituted with from 0 to 3 substituents independently chosen from hydroxy, halogen, cyano, nitro, C₁-C₆alkyl, (C₃-C₈cycloalkyl)C₀-C₄alkyl, C₁-C₄alkoxy, C₁-C₄alkoxycarbonyl, C₁-C₂haloalkyl and C₁-C₂haloalkoxy; and
each n is independently 0 or 1.

14. A method according to claim 7, wherein the compound has the formula: 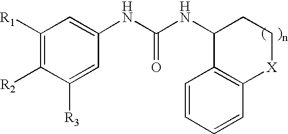
wherein X is CH, N or O; and n is 0, 1 or 2.

15. A method according to claim 1, wherein the compound is:
N-(4-butylphenyl)-N′-(1-phenylpropyl)urea;
N-(4-ethylphenyl)-N′-(2-phenylethyl)urea;
N-(4-ethylphenyl)-N′-(2-phenylethyl)urea;
N-(3-cyclohexyl-2-phenylpropyl)-N′-[4-(trifluoromethyl)phenyl]urea;
N-[4-(difluoromethoxy)phenyl]-N′-(2-methoxybenzyl)urea;
N-[4-(difluoromethoxy)phenyl]-N′-(4-methoxybenzyl)urea;
N-[4-(difluoromethoxy)benzyl]-N′-[4-(difluoromethoxy)phenyl]urea;
N-[4-(difluoromethoxy)phenyl]-N′-[1-(4-hydroxyphenyl)ethyl]urea;
N-[2-(2-chlorophenyl)ethyl]-N′-[4-(difluoromethoxy)phenyl]urea;
N-[2-(3-chlorophenyl)ethyl]-N′-[4-(difluoromethoxy)phenyl]urea;
N-[2-(4-chlorophenyl)ethyl]-N′-[4-(difluoromethoxy)phenyl]urea;
N-[4-(difluoromethoxy)phenyl]-N′-{2-[2-(trifluoromethyl)phenyl]ethyl}urea;
N-[4-(difluoromethoxy)phenyl]-N′-{2-[3-(trifluoromethyl)phenyl]ethyl}urea;
N-[4-(difluoromethoxy)phenyl]-N′-{2-[4-(trifluoromethyl)phenyl]ethyl}urea;
N-[4-(difluoromethoxy)phenyl]-N′-[2-(2-fluorophenyl)ethyl]urea;
N-[4-(di-fluoromethoxy)phenyl]-N′-[2-(4-fluorophenyl)ethyl]urea;
N-[2-(2,4-dichlorophenyl)ethyl]-N′-[4-(difluoromethoxy)phenyl]urea;
N-[4-(difluoromethoxy)phenyl]-N′-[2-(3-methoxyphenyl)ethyl]urea;
N-[4-(difluoromethoxy)phenyl]-N′-[2-(4-methoxyphenyl)ethyl]urea;
N-[4-(difluoromethoxy)phenyl]-N′-[2-(3-ethoxyphenyl)ethyl]urea;
N-[4-(difluoromethoxy)phenyl]-N′-[2-(2,4-dimethoxyphenyl)ethyl]urea;
N-[4-(difluoromethoxy)phenyl]-N′-[2-(3-fluorophenyl)ethyl]urea;
methyl 4-{[({[4-(difluoromethoxy)phenyl]amino}carbonyl)amino]methyl}benzoate;
tert-butyl 4-{[({[4-(difluoromethoxy)phenyl]amino}carbonyl)amino]methyl}piperidine-1-carboxylate;
N-[(3S)-1-benzylpyrrolidin-3-yl]-N′-[4-(difluoromethoxy)phenyl]urea;
N-[3-(difluoromethoxy)phenyl]-N′-(4-methoxybenzyl)urea;
methyl 4-{[({[3-(difluoromethoxy)phenyl]amino}carbonyl)amino]methyl}benzoate;
N-[3-(difluoromethoxy)phenyl]-N′-[2-(3-fluorophenyl)ethyl]urea;
N-[3-(difluoromethoxy)phenyl]-N′-(2-phenylcyclopropyl)urea;
N-[3-(difluoromethoxy)phenyl]-N′-[2-(3,5-difluorophenyl)ethyl]urea;
N-(3,4-dihydro-2H-chromen-4-yl)-N′-[3-(methylthio)phenyl]urea;
N-(2,3-dihydro-1H-inden-1-yl)-N′-[4-(methylthio)phenyl]urea;
N-[4-(methylthio)phenyl]-N′-(1,2,3,4-tetrahydronaphthalen-1-yl)urea;
N-(3,4-dihydro-2H-chromen-4-yl)-N′-[4-(methylthio)phenyl]urea;
N-[4-(methylthio)phenyl]-N′-(6,7,8,9-tetrahydro-5H-benzo[7]annulen-5-yl)urea;
N-(4-ethylphenyl)-N′-[2-(3-methylphenyl)ethyl]urea;
N-(4-ethylphenyl)-N′-[2-(3-fluorophenyl)ethyl]urea;
N-(4-ethylphenyl)-N′-[2-(4-fluorophenyl)ethyl]urea;
N-(3-fluoro-4-methylphenyl)-N′-[2-(3-fluorophenyl)ethyl]urea;
N-[2-(3-methylphenyl)ethyl]-N′-(4-nitrophenyl)urea;
N-[2-(2-fluorophenyl)ethyl]-N′-(4-nitrophenyl)urea;
N-[2-(3-fluorophenyl)ethyl]-N′-(4-nitrophenyl)urea;
N-[2-(4-fluorophenyl)ethyl]-N′-(4-nitrophenyl)urea;
N-[2-(3-methylphenyl)ethyl]-N′-[3-(methylthio)phenyl]urea;
N-[2-(2-fluorophenyl)ethyl]-N′-[3-(methylthio)phenyl]urea;
N-[2-(3-fluorophenyl)ethyl]-N′-[3-(methylthio)phenyl]urea;
N-[2-(4-fluorophenyl)ethyl]-N′-[3-(methylthio)phenyl]urea;
N-[2-(3-methylphenyl)ethyl]-N′-[4-(methylthio)phenyl]urea;
N-[2-(2-fluorophenyl)ethyl]-N′-[4-(methylthio)phenyl]urea;
N-[2-(3-fluorophenyl)ethyl]-N′-[4-(methylthio)phenyl]urea;
N-[2-(4-fluorophenyl)ethyl]-N′-[4-(methylthio)phenyl]urea;
N-(4-methylphenyl)-N′-(2-phenyloctyl)urea;
N-4-fluorophenyl)-N′-(2-phenyloctyl)urea;
N-(4-cyanophenyl)-N′-(2-phenyloctyl)urea;
N-(3-cyanophenyl)-N′-(2-phenyl octyl)urea;
N-(3-cyclopentyl-2-phenylpropyl)-N′-(4-nitrophenyl)urea; or
N-(4-nitrophenyl)-N′-(2-phenylheptyl)urea.

16. (canceled)

17. A method according, to claim 1 claim 16, wherein the condition is obesity, bulimia, alcohol dependency or nicotine dependency.

18. (canceled)

19. A pharmaceutical composition, comprising:
(i) a first agent that is a compound of the formula: 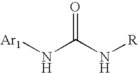
or a pharmaceutically acceptable salt thereof, wherein:
R is:
(a) (5- or 6-membered heteroaryl)C₁-C₆alkyl or phenylC₁-C₆alkyl, each of which is substituted with from 0 to 4 substituents that are independently chosen from Rₓ; or
(b) (C₃-C₈cycloalkyl)C₀-C₆alkyl or (3- to 8-membered heterocycloalkyl)C₀-C₆alkyl, each of which is
(i) optionally fused to a 5- to 7-membered carbocyclic or heterocyclic ring, and
(ii) substituted with from 0 to 4 substituents that are independently chosen from Rₓ;
Ar₁ is phenyl, naphthyl or a 5- to 10-membered heteroaryl, each of which is substituted with from 0 to 4 substituents that are independently chosen from R_{y};
Each Rₓ is independently:
(a) hydroxy, halogen, amino, cyano, nitro, aminocarbonyl, aminosulfonyl or —COOH;
(b) a group of the formula L-M₁-Q₁, wherein:
L is absent or C₀-C₄alkylene;
M₁ is absent, O, OC(═O), OC(═O)O, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen, C₁-C₈alkyl and groups that are taken together with Q₁ to form an optionally substituted 4- to 7-membered heterocycle; and
Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-1-membered heterocycle, each of which is substituted with from 0 to 3 substituents independently chosen from hydroxy, halogen, amino, cyano, oxo, C₁-C₄alkyl, C₁-C₄alkoxy and C₁-C₄haloalkyl;
such that R₁ is not unsubstituted benzyl; or
(c) taken together with an Rₓ located on an adjacent ring carbon atom to form a fused 5- to 7-membered carbocycle or heterocycle that is substituted with from 0 to 3 substituents independently chosen from hydroxy, halogen, amino, cyano, oxo, C₁-C₄alkyl, C₁-C₄alkoxy, C₁-C₄haloalkyl and C₁-C₄alkylsulfonyl; and
Each R_{y} is independently:
(a) hydroxy, halogen, amino, cyano, nitro, aminocarbonyl, aminosulfonyl or —COOH;
(b) a group of the formula L-M₂-Q₂, wherein:
L is absent or C₀-C₄alkylene;
M₂ is absent, O, C(═O), OC(═O), C(═O)O, O—C(═O)O, S(O)ₘ, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen and C₁-C₈alkyl; and
Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl;
(ii) a second agent that is suitable for treating an appetite disorder, obesity, an addictive disorder, asthma, liver cirrhosis, sepsis, irritable bowel disease, Crohn's disease, depression, schizophrenia, a memory disorder, a cognitive disorder, a movement disorder or bone loss; and
(iii) a physiologically acceptable carrier or excipient.

20. A pharmaceutical composition according to claim 19, wherein the second agent is an anti-obesity agent selected from an MCH receptor antagonist, an apo-B/MTP inhibitor, a 11β-hydroxy steroid dehydrogenase-1 inhibitor, peptide YY₃-36 or an analog thereof, a MCR-4 agonist, a CCK-A agonist, a monoamine reuptake inhibitor, a sympathomimetic agent, a β₃ adrenergic receptor agonist, a dopamine agonist, a melanocyte-stimulating hormone receptor analog, a 5-HT2c receptor agonist, leptin or an analog thereof, a leptin receptor agonist, a galanin antagonist, a lipase inhibitor, a bombesin agonist, a neuropeptide-Y receptor antagonist, a thyromimetic agent, dehydroepiandrosterone or analog thereof, a glucocorticoid receptor antagonist, an orexin receptor antagonist, a glucagon-like peptide-1 receptor agonist, a ciliary neurotrophic factor, a human agouti-related protein antagonist, a ghrelin receptor antagonist, a histamine 3 receptor antagonist, or a neuromedin U receptor agonist.

21. A pharmaceutical composition according to claim 20, wherein the anti-obesity agent is phentermine, orlistat or sibutramine.

22. A pharmaceutical composition according to claim 19, wherein the second agent is a nicotine receptor partial agonist, an opioid antagonist or a dopaminergic agent.

23. A pharmaceutical composition according to claim 19, wherein the second agent is suitable for treating an addictive disorder, and wherein the agent is selected from methadone, LAAM, naltrexone, ondansetron, sertraline, fluoxetine, diazepam, chlordiazepoxide, varenicline and bupropion.

24. A pharmaceutical composition according to claim 19, wherein Ar₁ is phenyl, pyridyl or pyrimidyl, each of which is substituted with from 0 to 3 substituents independently located meta or para to the point of attachment, wherein each substituent is independently:
(a) halogen, hydroxy or cyano; or
(b) C₁-C₄alkyl, C₁-C₄alkoxy, C₁-C₄alkylthio, C₁-C₄haloalkyl, C₁-C₄haloalkoxy, C₁-C₄alkanoyl, C₁-C₄alkanoyloxy or C₁-C₄alkoxycarbonyl, each of which is substituted with from 0 to 2 substituents independently chosen from hydroxy, halogen, C₁-C₄alkyl, C₁-C₄alkoxy, C₂-C₄alkanoyl, and C₁-C₄haloalkyl.

25. A pharmaceutical composition according to claim 19, wherein Ar₁ is phenyl that is unsubstituted or substituted with 1 or 2 substituents, each of which is located meta or para to the point of attachment, and each of which is independently halogen, cyano, nitro, C₁-C₄alkyl, C₁-C₄alkoxy, C₁-C₄alkylthio, C₁-C₄haloalkyl or C₁-C₄haloalkoxy.

26. A pharmaceutical composition according to claim 19, wherein R is phenylC₁-C₄alkyl that is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

27. A pharmaceutical composition according to claim 19, wherein R is (C₃-C₈cycloalkyl)C₀-C₄alkyl or (3- to 8-membered heterocycloalkyl)C₀-C₄alkyl, each of which is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl, C₁-C₂haloalkoxy, phenylC₀-C₄alkyl and (6-membered heteroaryl)C₀-C₄alkyl.

28. A pharmaceutical composition according to claim 19, wherein R is (C₃-C₈cycloalkyl)C₀-C₄alkyl or (3- to 8-membered heterocycloalkyl)C₀-C₄alkyl, each of which is fused to a phenyl or 6-membered heteroaryl ring and substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

29. A pharmaceutical composition according to claim 25, wherein the compound has the formula: 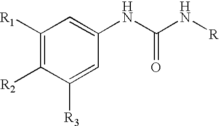
wherein R₁, R₂ and R₃ are independently chosen from hydrogen and R_{y}, and wherein at least one of R₁ and R₂ is not hydrogen.

30. A packaged pharmaceutical preparation, comprising:
(i) pharmaceutical composition comprising a compound of the formula: 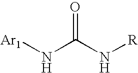
or a pharmaceutically acceptable salt thereof, wherein:
R is:
(a) (5- or 6-membered heteroaryl)C₁-C₆alkyl or phenylC₁-C₆alkyl, each of which is substituted with from 0 to 4 substituents that are independently chosen from Rₓ; or
(b) (C₃-C₈cycloalkyl)C₀-C₆alkyl or (3- to 8-membered heterocycloalkyl)C₀-C₆alkyl, each of which is
(i) optionally fused to a 5- to 7-membered carbocyclic or heterocyclic ring, and
(ii) substituted with from 0 to 4 substituents that are independently chosen from R_{y};
Ar₁ is phenyl, naphthyl or a 5- to 10-membered heteroaryl, each of which is substituted with from 0 to 4 substituents that are independently chosen from R_{y};
Each Rₓ is independently:
(a) hydroxy, halogen, amino, cyano, nitro, aminocarbonyl, aminosulfonyl or —COOH;
(b) a group of the formula L-M₁-Q₁, wherein:
L is absent or C₀-C₄alkylene;
M₁ is absent, O, OC(═O), OC(═O)O, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen, C₁-C₈alkyl and groups that are taken together with Q₁ to form an optionally substituted 4- to 7-membered heterocycle; and
Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-membered heterocycle, each of which is substituted with from 0 to 3 substituents independently chosen from hydroxy, halogen, amino, cyano, oxo, C₁-C₄alkyl, C₁-C₄alkoxy and C₁-C₄haloalkyl;
such that Rₓ is not unsubstituted benzyl; or
(c) taken together with an Rₓ located on an adjacent ring carbon atom to form a fused 5- to 7-membered carbocycle or heterocycle that is substituted with from 0 to 3 substituents independently chosen from hydroxy, halogen, amino, cyano, oxo, C₁-C₄alkyl, C₁-C₄alkoxy, C₁-C₄haloalkyl and C₁-C₄alkylsulfonyl; and
Each R_{y} is independently:
(a) hydroxy, halogen, amino, cyano, nitro, aminocarbonyl, aminosulfonyl or —COOH;
(b) a group of the formula L-M₂-Q₂, wherein:
L is absent or C₀-C₄alkylene;
M₂ is absent, O, C(═O), OC(═O), C(O)O, O—C(═O)O, S(O)ₘ, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen and C₁-C₈alkyl; and
Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl;
in combination with a physiologically acceptable carrier or excipient; and
(ii) instructions for using the composition to treat an appetite disorder, obesity, an addictive disorder, asthma, liver cirrhosis, sepsis, irritable bowel disease, Crohn's disease, depression, schizophrenia, a memory disorder, a cognitive disorder, a movement disorder or bone loss.

31-36. (canceled)

37. A method for identifying a non-competitive CB1 antagonist, comprising:
(i) contacting CB1 with a labeled, non-competitive CB1 antagonist and a test compound, under conditions that permit binding of the CB1 antagonist to CB1; wherein the CB1 antagonist is a compound of the formula: 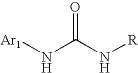
or a pharmaceutically acceptable salt thereof, wherein:
R is:
(a) (5- or 6-membered heteroaryl)C₁-C₆alkyl or phenylC₁-C₆alkyl, each of which is substituted with from 0 to 4 substituents that are independently chosen from Rₓ; or
(b) (C₃-C₈cycloalkyl)C₀-C₆alkyl or (3- to 8-membered heterocycloalkyl)C₀-C₆alkyl, each of which is
(i) optionally fused to a 5- to 7-membered carbocyclic or heterocyclic ring, and
(ii) substituted with from 0 to 4 substituents that are independently chosen from Rₓ;
Ar₁ is phenyl, naphthyl or a 5- to 10-membered heteroaryl, each of which is substituted with from to 4 substituents that are independently chosen from R_{y};
Each Rₓ is independently:
(a) hydroxy, halogen, amino, cyano, nitro, aminocarbonyl, aminosulfonyl or —COOH;
(b) a group of the formula L-M₁-Q₁, wherein:
L is absent or C₀-C₄alkylene;
M₁ is absent, O, OC(═O), OC(═O)O, N(R_{z}), C(═O)N(R_{z}), C(═NH)N(R_{z}), N(R)C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen, C₁-C₈alkyl and groups that are taken together with Q₁ to form an optionally substituted 4- to 7-membered heterocycle; and
Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-membered heterocycle, each of which is substituted with from 0 to 3 substituents independently chosen from hydroxy, halogen, amino, cyano, oxo, C₁-C₄alkyl, C₁-C₄alkoxy and C₁-C₄haloalkyl;
such that Rₓ is not unsubstituted benzyl; or
(c) taken together with an Rₓ located on an adjacent ring carbon atom to form a fused 5- to 7-membered carbocycle or heterocycle that is substituted with from 0 to 3 substituents independently chosen from hydroxy, halogen, amino, cyano, OXO, C₁-C₄alkyl, C₁-C₄alkoxy, C₁-C₄haloalkyl and C₁-C₄alkylsulfonyl; and
Each R_{y} is independently:
(a) hydroxy, halogen, amino, cyano, nitro, aminocarbonyl, aminosulfonyl or —COOH;
(b) a group of the formula L-M₂-Q₂, wherein:
L is absent or C₀-C₄alkylene;
M₂ is absent, O, C(═O), OC(═O), C(═O)O, O—C(═O)O, S(O)ₘ, N(R_{z}), C(═O)N(R_{z}), C(═NH)N(R_{z}), N(R_{z})C(═O), N(R_{z})C(NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen and C₁-C₈alkyl; and
Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl;
(ii) removing unbound labeled, non-competitive CB1 antagonist and test compound;
(iii) detecting a signal that corresponds to the amount of labeled, non-competitive CB1 antagonist bound to the CB1; and
(iv) comparing the signal to a reference signal that corresponds to the amount of labeled, non-competitive CB1 antagonist bound to the CB1 in the absence of test compound;
and therefrom identifying a non-competitive CB1 antagonist.

38. A method for suppressing appetite in a patient, comprising administering to the patient an appetite reducing amount of at least one compound of the formula: 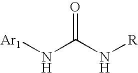
or a pharmaceutically acceptable salt thereof, wherein:
R is:
(a) (5- or 6-membered heteroaryl)C₁-C₆alkyl or phenylC₁-C₆alkyl, each of which is substituted with from 0 to 4 substituents that are independently chosen from Rₓ; or
(b) (C₃-C₈cycloalkyl)C₀-C₆alkyl or (3- to 8-membered heterocycloalkyl)C₀-C₆alkyl, each of which is
(i) optionally fused to a 5- to 7-membered carbocyclic or heterocyclic ring, and
(ii) substituted with from 0 to 4 substituents that are independently chosen from Rₓ;
Ar₁ is phenyl, naphthyl or a 5- to 10-membered heteroaryl, each of which is substituted with from 0 to 4 substituents that are independently chosen from R_{y};
Each Rₓ is independently:
(a) hydroxy, halogen, amino, cyano, nitro, aminocarbonyl, aminosulfonyl or —COOH;
(b) a group of the formula L-M₁-Q₁, wherein:
L is absent or C₀-C₄alkylene;
M₁ is absent, O, OC(═O), OC(O)O, N(R_{z}), C(═O)N(R_{z}), C(═NH)N(R_{z}), N(R_{z})C(═O), N(R_{z})C(═NH), N(R_{z})C(═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R_{z} is independently selected at each occurrence from hydrogen, C₁-C₈alkyl and groups that are taken together with Q₁ to form an optionally substituted 4- to 7-membered heterocycle; and
Q₁ is C₁-C₈alkyl, (C₃-C₈cycloalkyl)C₀-C₄alkyl, phenylC₀-C₄alkyl, (5- to 10-membered heterocycle)C₀-C₄alkyl or taken together with M₁ to form a 4- to 7-membered heterocycle, each of which is substituted with from 0 to 3 substituents independently chosen from hydroxy, halogen, amino, cyano, oxo, C₁-C₄alkyl, C₁-C₄alkoxy and C₁-C₄haloalkyl;
such that Rₓ is not unsubstituted benzyl; or
(c) taken together with an Rₓ located on an adjacent ring carbon atom to form a fused 5- to 7-membered carbocycle or heterocycle that is substituted with from 0 to 3 substituents independently chosen from hydroxy, halogen, amino, cyano, oxo, C₁-C₄alkyl, C₁-C₄alkoxy, C₁-C₄haloalkyl and C₁-C₄alkylsulfonyl; and
Each R_{y} is independently:
(a) hydroxy, halogen, amino, cyano, nitro, aminocarbonyl, aminosulfonyl or —COOH;
(b) a group of the formula L-M₂-Q₂, wherein:
L is absent or C₀-C₄alkylene;
M₂ is absent, O, C(═O), OC(═O), C(═O)O, O—C(═O)O, S(O)ₘ, N(R_{z}), C(═O)N(R_{z}), C(═NH)N(R_{z}), N(R_{z})C(═O), N(R_{z})C(═NH), N(R_{z})C═O)O, OC(═O)N(R_{z}), N(R_{z})S(O)ₘ, S(O)ₘN(R_{z}) or N[S(O)ₘR_{z}]S(O)ₘ; wherein m is independently selected at each occurrence from 0, 1 and 2; and R₁ is independently selected at each occurrence from hydrogen and C₁-C₈alkyl; and
Q₂ is C₁-C₈alkyl or (C₃-C₈cycloalkyl)C₀-C₄alkyl, each of which is substituted with from 0 to 3 substituents independently chosen from hydroxy, halogen, amino, cyano, oxo, C₁-C₄alkyl, C₁-C₄alkoxy and C₁-C₄haloalkyl;
and thereby suppressing appetite in the patient.

39. A method according to claim 38, wherein Ar₁ is phenyl, pyridyl or pyrimidyl, each of which is substituted with from 0 to 3 substituents independently located meta or para to the point of attachment, wherein each substituent is independently:
(a) halogen, hydroxy or cyano; or
(b) C₁-C₄alkyl, C₁-C₄alkoxy, C₁-C₄alkylthio, C₁-C₄haloalkyl, C₁-C₄haloalkoxy, C₁-C₄alkanoyl, C₁-C₄alkanoyloxy or C₁-C₄alkoxycarbonyl, each of which is substituted with from 0 to 2 substituents independently chosen from hydroxy, halogen, C₁-C₄alkyl, C₁-C₄alkoxy, C₂-C₄alkanoyl, and C₁-C₄haloalkyl.

40. A method according to claim 39, wherein Ar₁ is phenyl that is unsubstituted or substituted with 1 or 2 substituents, each of which is located meta or para to the point of attachment, and each of which is independently halogen, cyano, nitro, C₁-C₄alkyl, C₁-C₄alkoxy, C₁-C₄alkylthio, C₁-C₄haloalkyl or C₁-C₄haloalkoxy.

41. A method according to claim 38, wherein R is phenylC₁-C₄alkyl that is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

42. A method according to claim 38, wherein R is (C₃-C₈cycloalkyl)C₀-C₄alkyl or (3- to 8-membered heterocycloalkyl)C₀-C₄alkyl, each of which is substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl, C₁-C₂haloalkoxy, phenylC₀-C₄alkyl and (6-membered heteroaryl)C₀-C₄alkyl.

43. A method according to claim 38, wherein R is (C₃-C₈cycloalkyl)C₀-C₄alkyl or (3- to 8-membered heterocycloalkyl)C₀-C₄alkyl, each of which is fused to a phenyl or 6-membered heteroaryl ring and substituted with from 0 to 3 substituents independently chosen from halogen, C₁-C₆alkyl, (C₃-C₈cycloalkyl)C₀-C₄alkyl, C₁-C₄alkoxy, C₁-C₄alkoxycarbonyl and C₁-C₂haloalkoxy.

44. A method according to claim 38, wherein the compound has the formula: 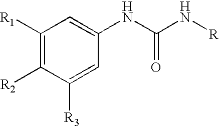
wherein R₁, R₂ and R₃ are independently chosen from hydrogen and R_{y}, and wherein at least one of R₁ and R₂ is not hydrogen.
